SEQUENCE LISTING (126 sequences)

SEQ ID NO: 1 - DNA, 1770 bp, Solanum lycopersicum
  source 1..1770: mol_type = genomic DNA
atgaaggctttgagtatagccgtagcagcattttcaacattccatacaactcgccctcgtctcttcactttcccaaacccttctacttcttcttctctccttcttccgtccttcaaactaaagagattttctatcttctccattcaacaaccgccgccggcgttagggttttacgccggagttacaacaatggtggagggctcagcatttcagccgttgtcagttcaagtgaaggaccatattgatttatcgccaaaagaggagcagatattcaaccgtcttcttcaagttgtcgagcattataatatgggaacacagcttcgtgttgctggtggttgggttcgcgataagctgctaggtaaagagtgctatgatattgatattgctcttgacaacatgtatggacgagaattctgtgagaaagtgaacgagtacttgtcagcatcaggtgaggaaacccaaggagttggtgttatccagtgcaatcccgatcagtcaaaacacttggaaacagcgaggatgcgccttttcgacgtatggattgattttgtcaacttaagagcagaagactacagtgagaacagtcgaattccaacaatgaaatttgggactgcaaaacaggatgctgatcgcagggatttaaccattaatagcttgttttacaacattaacaccagatcagtcgaagatcttacagaaagaggcattgcagatctgaagtctggaaaaattgtcactccactacctccaaagcagacctttttggatgatcccctaagagttcttcgagctattcgttttggtgcaaggtttgggtttatattagatgaagaactaaagaaggcagctgcagatgaagacgtgagagttgctattgctgataaaattagcagagaacgcataggacatgaaattgatcttatgatgtctggtgaccaaccggtcaaagcaatgacttatattgctgacttgaagttattttcggctgtatttagtcttccttctgattttgagccatcaattcctgatggatgtgataggctgtgtgttacttgcttgggttcagcttgggaacttttaaaccttattggacgctcctcattttcagatgatcagaggagactttgcctgtatgctgctttatttttgccattcagagagaccatttatagagataacaaagccaaaaagattccagttgtcaattatatttttcgcaattctctcaagttgaaggccagtgatgcggaaatagtcatgagcttgcatacggtaactaggaagtttgtcaccttaatatcactactcatttctaaagacgatatccaagttcttgaagttgattggaaaagggataccattgaggttccaatagcttcaaaacttagaatattgactggtttgctacttcgagaaattaaagaattttggcgtgtcgctcttcttctatctatgctgctacatcctgttgacgttgtgtcctctacaagtttctcaaatgagaactttgaactggacaagagtagcggattatttaagacagtagagaatgcagtaagaacactaggtctggagaaggtttgggaaatgaaaccactggttgatggaaaggaaattatgaatattttggagattaaaagtggagggccagttgttagtgaatggcaacaaaaactgctggagtggcagctcgcacacccttctggttcagctgaagaatgcatcgactggatgaatcaagcacagtcaaaacgtgcaagaaccgagtag

SEQ ID NO: 2 - protein, 589 aa, Solanum lycopersicum
MKALSIAVAAFSTFHTTRPRLFTFPNPSTSSSLLLPSFKLKRFSIFSIQQPPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLFDVWIDFVNLRAEDYSENSRIPTMKFGTAKQDADRRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVTCLGSAWELLNLIGRSSFSDDQRRLCLYAALFLPFRETIYRDNKAKKIPVVNYIFRNSLKLKASDAEIVMSLHTVTRKFVTLISLLISKDDIQVLEVDWKRDTIEVPIASKLRILTGLLLREIKEFWRVALLLSMLLHPVDVVSSTSFSNENFELDKSSGLFKTVENAVRTLGLEKVWEMKPLVDGKEIMNILEIKSGGPVVSEWQQKLLEWQLAHPSGSAEECIDWMNQAQSKRARTE

SEQ ID NO: 3 - DNA, 1770 bp, Solanum lycopersicum
  source 1..1770: mol_type = genomic DNA
atgaaggctttgagtatagccgtagcagcattttcaacattccatacaactcgccctcgtctcttcactttcccaaacccttctacttcttcttctctccttcttccgtccttcaaactaaagagattttctatcttctccattcaacaaccgccgccggcgttagggttttacgccggagttacaacaatggtggagggctcagcatttcagccgttgtcagttcaagtgaaggaccatattgatttatcgccaaaagaggagcagatattcaaccgtcttcttcaagttgtcgagcattataatatgggaacacagcttcgtgttgctggtggttgggttcgcgataagctgctaggtaaagagtgctatgatattgatattgctcttgacaacatgtatggacgagaattctgtgagaaagtgaacgagtacttgtcagcatcaggtgaggaaacccaaggagttggtgttatccagtgcaatcccgatcagtcaaaacacttggaaacagcgaggatgcgccttttcgacgtatggattgattttgtcaacttaagagcagaagactacagtgagaacagtcgaattccaacaatgaaatttgggactgcaaaacaggatgctgatcgcagggatttaaccattaatagcttgttttacaacattaacaccagatcagtcgaagatcttacagaaagaggcattgcagatctgaagtctggaaaaattgtcactccactacctccaaagcagacctttttggatgatcccctaagagttcttcgagctattcgttttggtgcaaggtttgggtttatattagatgaagaactaaagaaggcagctgcagatgaagacgtgagagttgctattgctgataaaattagcagagaacgcataggacatgaaattgatcttatgatgtctggtgaccaaccggtcaaagcaatgacttatattgctgacttgaagttattttcggctgtatttagtcttccttctgattttgagccatcaattcctgatggatgtgataggctgtgtgttacttgcttgggttcagcttgggaacttttaaaccttattggacgctcctcattttcagatgatcagaggagactttgcctgtatgctgctttatttttgccattcagagagaccatttatagagataacaaagccaaaaagattccagttgtcaattatatttttcgcaattctctcaagttgaaggccagtgatgcggaaatagtcatgagcttgcatacggtaactaggaaatttgtcaccttaatatcactactcatttctaaagacgatatccaagttcttgaagttgattggaaaagggataccattgaggttccaatagcttcaaaacttagaatattgactggtttgctacttcgagaaattaaagaattttggcgtgtcgctcttcttctatctatgctgctacatcctgttgacgttgtgtcctctacaagtttctcaaatgagaactttgaactggacaagagtagcggattatttaagacagtagagaatgcagtaagaacactaggtctggagaaggtttgggaaatgaaaccactggttaatggaaaggaaattatgaatattttggagattaaaagtggagggccagttgttagagaatggcaacaaaaactgctggagtggcagctcgcacacccttctggttcagctgaagaatgcatcgactggatgaagcaagcacagtcaaaacgtgcaagaaccgagtag

SEQ ID NO: 4 - protein, 589 aa, Solanum lycopersicum
MKALSIAVAAFSTFHTTRPRLFTFPNPSTSSSLLLPSFKLKRFSIFSIQQPPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLFDVWIDFVNLRAEDYSENSRIPTMKFGTAKQDADRRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVTCLGSAWELLNLIGRSSFSDDQRRLCLYAALFLPFRETIYRDNKAKKIPVVNYIFRNSLKLKASDAEIVMSLHTVTRKFVTLISLLISKDDIQVLEVDWKRDTIEVPIASKLRILTGLLLREIKEFWRVALLLSMLLHPVDVVSSTSFSNENFELDKSSGLFKTVENAVRTLGLEKVWEMKPLVNGKEIMNILEIKSGGPVVREWQQKLLEWQLAHPSGSAEECIDWMKQAQSKRARTE

SEQ ID NO: 5-12 - skipped (8 entries)

SEQ ID NO: 13 - DNA, 1800 bp, Solanum lycopersicum
  source 1..1800: mol_type = genomic DNA
catcacttgtccgttagaaagagagaagaaatgaaggctttgagtatagccgtagcagcattttcaacattccatacaactcgccctcgtctcttcactttcccaaacccttctacttcttcttctctccttcttccgtccttcaaactaaagagattttctatcttctccattcaacaaccgccgccggcgttagggttttacgccggagttacaacaatggtggagggctcagcatttcagccgttgtcagttcaagtgaaggaccatattgatttatcgccaaaagaggagcagatattcaaccgtcttcttcaagttgtcgagcattataatatgggaacacagcttcgtgttgctggtggttgggttcgcgataagctgctaggtaaagagtgctatgatattgatattgctcttgacaacatgtatggacgagaattctgtgagaaagtgaacgagtacttgtcagcatcaggtgaggaaacccaaggagttggtgttatccagtgcaatcccgatcagtcaaaacacttggaaacagcgaggatgcgccttttcgacgtatggattgattttgtcaacttaagagcagaagactacagtgagaacagtcgaattccaacaatgaaatttgggactgcaaaacaggatgctgatcgcagggatttaaccattaatagcttgttttacaacattaacaccagatcagtcgaagatcttacagaaagaggcattgcagatctgaagtctggaaaaattgtcactccactacctccaaagcagacctttttggatgatcccctaagagttcttcgagctattcgttttggtgcaaggtttgggtttatattagatgaagaactaaagaaggcagctgcagatgaagacgtgagagttgctattgctgataaaattagcagagaacgcataggacatgaaattgatcttatgatgtctggtgaccaaccggtcaaagcaatgacttatattgctgacttgaagttattttcggctgtatttagtcttccttctgattttgagccatcaattcctgatggatgtgataggctgtgtgttacttgcttgggttcagcttgggaacttttaaaccttattggacgctcctcattttcagatgatcagaggagactttgcctgtatgctgctttatttttgccattcagagagaccatttatagagataacaaagccaaaaagattccagttgtcaattatatttttcgcaattctctcaagttgaaggccagtgatgcggaaatagtcatgagcttgcatacggtaactaggaaatttgtcaccttaatatcactactcatttctaaagacgatatccaagttcttgaagttgattggaaaagggataccattgaggttccaatagcttcaaaacttagaatattgactggtttgctacttcgagaaattaaagaattttggcgtgtcgctcttcttctatctatgctgctacatcctgttgacgttgtgtcctctacaagtttctcaaatgagaactttgaactggacaagagtagcggattatttaagacagtagagaatgcagtaagaacactaggtctggagaaggtttgggaaatgaaaccactggttaatggaaaggaaattatgaatattttggagattaaaagtggagggccagttgttagagaatggcaacaaaaactgctggagtggcagctcgcacacccttctggttcagctgaagaatgcatcgactggatgaagcaagcacagtcaaaacgtgcaagaaccgagtag

SEQ ID NO: 14 - protein, 599 aa, Solanum lycopersicum
HHLSVRKREEMKALSIAVAAFSTFHTTRPRLFTFPNPSTSSSLLLPSFKLKRFSIFSIQQPPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLFDVWIDFVNLRAEDYSENSRIPTMKFGTAKQDADRRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVTCLGSAWELLNLIGRSSFSDDQRRLCLYAALFLPFRETIYRDNKAKKIPVVNYIFRNSLKLKASDAEIVMSLHTVTRKFVTLISLLISKDDIQVLEVDWKRDTIEVPIASKLRILTGLLLREIKEFWRVALLLSMLLHPVDVVSSTSFSNENFELDKSSGLFKTVENAVRTLGLEKVWEMKPLVNGKEIMNILEIKSGGPVVREWQQKLLEWQLAHPSGSAEECIDWMKQAQSKRARTE

SEQ ID NO: 15-18 - skipped (4 entries)

SEQ ID NO: 19 - DNA, 22 bp, synthetic construct
  misc_feature 1..22: note = Single strand DNA oligonucleotide
  source 1..22: mol_type = other DNA
gggagaattgaatgtggagggt

SEQ ID NO: 20 - DNA, 20 bp, synthetic construct
  misc_feature 1..20: note = Single strand DNA oligonucleotide
  source 1..20: mol_type = other DNA
gacaggttcgtgattgcagc

SEQ ID NO: 21 - DNA, 24 bp, synthetic construct
  misc_feature 1..24: note = Single strand DNA oligonucleotide
  source 1..24: mol_type = other DNA
gcagatttagaggtcagatccttc

SEQ ID NO: 22 - DNA, 24 bp, synthetic construct
  misc_feature 1..24: note = Single strand DNA oligonucleotide
  source 1..24: mol_type = other DNA
catgccagtaccagagttcaatag

SEQ ID NO: 23 - DNA, 25 bp, synthetic construct
  misc_feature 1..25: note = Single strand DNA oligonucleotide
  source 1..25: mol_type = other DNA
tcactctcaaagcaattcataatgt

SEQ ID NO: 24 - DNA, 20 bp, synthetic construct
  misc_feature 1..20: note = Single strand DNA oligonucleotide
  source 1..20: mol_type = other DNA
gagcttcagtgggtctcaat

SEQ ID NO: 25 - DNA, 25 bp, synthetic construct
  misc_feature 1..25: note = Single strand DNA oligonucleotide
  source 1..25: mol_type = other DNA
atcctaccaactccgagaatagaac

SEQ ID NO: 26 - DNA, 25 bp, synthetic construct
  misc_feature 1..25: note = Single strand DNA oligonucleotide
  source 1..25: mol_type = other DNA
ggtgtggaatctagcaacataaatc

SEQ ID NO: 27 - DNA, 22 bp, synthetic construct
  misc_feature 1..22: note = Single strand DNA oligonucleotide
  source 1..22: mol_type = other DNA
cccatgggtctttttctctttc

SEQ ID NO: 28 - DNA, 24 bp, synthetic construct
  misc_feature 1..24: note = Single strand DNA oligonucleotide
  source 1..24: mol_type = other DNA
aatacatgttgggtcctatgtgtg

SEQ ID NO: 29 - DNA, 23 bp, synthetic construct
  misc_feature 1..23: note = Single strand DNA oligonucleotide
  source 1..23: mol_type = other DNA
ggtaccctctcaatctcaaggtc

SEQ ID NO: 30 - DNA, 22 bp, synthetic construct
  misc_feature 1..22: note = Single strand DNA oligonucleotide
  source 1..22: mol_type = other DNA
gaatttacacgccaccttcctc

SEQ ID NO: 31 - DNA, 23 bp, synthetic construct
  misc_feature 1..23: note = Single strand DNA oligonucleotide
  source 1..23: mol_type = other DNA
ttcttccattatcacaccatggg

SEQ ID NO: 32 - DNA, 20 bp, synthetic construct
  misc_feature 1..20: note = Single strand DNA oligonucleotide
  source 1..20: mol_type = other DNA
ttgtttgctcacaggcacac

SEQ ID NO: 33 - DNA, 25 bp, synthetic construct
  misc_feature 1..25: note = Single strand DNA oligonucleotide
  source 1..25: mol_type = other DNA
tcgagagaggagaggttataaggac

SEQ ID NO: 34 - DNA, 22 bp, synthetic construct
  misc_feature 1..22: note = Single strand DNA oligonucleotide
  source 1..22: mol_type = other DNA
gacggggtattccttggttatc

SEQ ID NO: 35 - DNA, 24 bp, synthetic construct
  misc_feature 1..24: note = Single strand DNA oligonucleotide
  source 1..24: mol_type = other DNA
agaactactgcctcgagtttcttc

SEQ ID NO: 36 - DNA, 25 bp, synthetic construct
  misc_feature 1..25: note = Single strand DNA oligonucleotide
  source 1..25: mol_type = other DNA
actctcgagtctagacactcattgg

SEQ ID NO: 37 - DNA, 20 bp, synthetic construct
  misc_feature 1..20: note = Single strand DNA oligonucleotide
  source 1..20: mol_type = other DNA
ctcgcgtccaatggtttgag

SEQ ID NO: 38 - DNA, 23 bp, synthetic construct
  misc_feature 1..23: note = Single strand DNA oligonucleotide
  source 1..23: mol_type = other DNA
tgcaccttcaattaacgtgagac

SEQ ID NO: 39 - DNA, 25 bp, synthetic construct
  misc_feature 1..25: note = Single strand DNA oligonucleotide
  source 1..25: mol_type = other DNA
gacttgagtaactgggaaactggac

SEQ ID NO: 40 - DNA, 22 bp, synthetic construct
  misc_feature 1..22: note = Single strand DNA oligonucleotide
  source 1..22: mol_type = other DNA
ggtgcccaaagtatgatgaatg

SEQ ID NO: 41 - DNA, 22 bp, synthetic construct
  misc_feature 1..22: note = Single strand DNA oligonucleotide
  source 1..22: mol_type = other DNA
gaagaagaagcaggccagaaag

SEQ ID NO: 42 - DNA, 22 bp, synthetic construct
  misc_feature 1..22: note = Single strand DNA oligonucleotide
  source 1..22: mol_type = other DNA
gagatagccgagcgatattgag

SEQ ID NO: 43 - DNA, 22 bp, synthetic construct
  misc_feature 1..22: note = Single strand DNA oligonucleotide
  source 1..22: mol_type = other DNA
tccaagtggcatgtttaatgac

SEQ ID NO: 44 - DNA, 22 bp, synthetic construct
  misc_feature 1..22: note = Single strand DNA oligonucleotide
  source 1..22: mol_type = other DNA
cgagttccaatactttcccatc

SEQ ID NO: 45 - DNA, 24 bp, synthetic construct
  misc_feature 1..24: note = Single strand DNA oligonucleotide
  source 1..24: mol_type = other DNA
gtcgtgacagaaatagatggaatg

SEQ ID NO: 46 - DNA, 24 bp, synthetic construct
  misc_feature 1..24: note = Single strand DNA oligonucleotide
  source 1..24: mol_type = other DNA
tacatctgaccctcatatgctagg

SEQ ID NO: 47 - DNA, 24 bp, synthetic construct
  misc_feature 1..24: note = Single strand DNA oligonucleotide
  source 1..24: mol_type = other DNA
cagagagttacacacgaccaagtc

SEQ ID NO: 48 - DNA, 24 bp, synthetic construct
  misc_feature 1..24: note = Single strand DNA oligonucleotide
  source 1..24: mol_type = other DNA
aggttatcttctttaacccccaag

SEQ ID NO: 49 - DNA, 29 bp, synthetic construct
  misc_feature 1..29: note = Single strand DNA oligonucleotide
  source 1..29: mol_type = other DNA
agactcgagatgaaggctttgagtatagc

SEQ ID NO: 50 - DNA, 30 bp, synthetic construct
  misc_feature 1..30: note = Single strand DNA oligonucleotide
  source 1..30: mol_type = other DNA
gacggtacctcgtccatacatgttgtcaag

SEQ ID NO: 51 - DNA, 29 bp, synthetic construct
  misc_feature 1..29: note = Single strand DNA oligonucleotide
  source 1..29: mol_type = other DNA
gactctagaatgaaggctttgagtatagc

SEQ ID NO: 52 - DNA, 30 bp, synthetic construct
  misc_feature 1..30: note = Single strand DNA oligonucleotide
  source 1..30: mol_type = other DNA
gacggatcctcgtccatacatgttgtcaag

SEQ ID NO: 53 - DNA, 20 bp, synthetic construct
  misc_feature 1..20: note = Guide RNA sequences (gRNA)
  source 1..20: mol_type = other DNA
gtaaacataacctttagtga

SEQ ID NO: 54 - DNA, 20 bp, synthetic construct
  misc_feature 1..20: note = Guide RNA sequences (gRNA)
  source 1..20: mol_type = other DNA
gagctggacaaaaggaacag

SEQ ID NO: 55 - DNA, 20 bp, synthetic construct
  misc_feature 1..20: note = Guide RNA sequences (gRNA)
  source 1..20: mol_type = other DNA
caaaacacttggaaacagcg

SEQ ID NO: 56 - DNA, 20 bp, synthetic construct
  misc_feature 1..20: note = Guide RNA sequences (gRNA)
  source 1..20: mol_type = other DNA
ttacttgcttgggttcagct

SEQ ID NO: 57 - DNA, 31 bp, synthetic construct
  misc_feature 1..31: note = Single strand DNA oligonucleotide
  source 1..31: mol_type = other DNA
tgtggtctcaagcgtaatgccaactttgtac

SEQ ID NO: 58 - DNA, 29 bp, synthetic construct
  misc_feature 1..29: note = Single strand DNA oligonucleotide
  source 1..29: mol_type = other DNA
gacgaattcatgaaggctttgagtatagc

SEQ ID NO: 59 - DNA, 30 bp, synthetic construct; identical to SEQ ID NO: 52
  misc_feature 1..30: note = Single strand DNA oligonucleotide
  source 1..30: mol_type = other DNA

SEQ ID NO: 60 - DNA, 25 bp, synthetic construct
  misc_feature 1..25: note = Single strand DNA oligonucleotide
  source 1..25: mol_type = other DNA
agatagccacaaggacaggaaatat

SEQ ID NO: 61 - DNA, 25 bp, synthetic construct
  misc_feature 1..25: note = Single strand DNA oligonucleotide
  source 1..25: mol_type = other DNA
ggccatatttcctgtccttgtggct

SEQ ID NO: 62 - DNA, 25 bp, synthetic construct
  misc_feature 1..25: note = Single strand DNA oligonucleotide
  source 1..25: mol_type = other DNA
agatgagacgatattcatgcatgat

SEQ ID NO: 63 - DNA, 25 bp, synthetic construct
  misc_feature 1..25: note = Single strand DNA oligonucleotide
  source 1..25: mol_type = other DNA
ggccatcatgcatgaatatcgtctc

SEQ ID NO: 64 - DNA, 27 bp, synthetic construct
  misc_feature 1..27: note = Single strand DNA oligonucleotide
  source 1..27: mol_type = other DNA
agatgcggtgaaatacaagaaaagtgc

SEQ ID NO: 65 - DNA, 27 bp, synthetic construct
  misc_feature 1..27: note = Single strand DNA oligonucleotide
  source 1..27: mol_type = other DNA
ggccgcacttttcttgtatttcaccgc

SEQ ID NO: 66 - DNA, 25 bp, synthetic construct
  misc_feature 1..25: note = Single strand DNA oligonucleotide
  source 1..25: mol_type = other DNA
agataccgccaaatgtgatatgaca

SEQ ID NO: 67 - DNA, 25 bp, synthetic construct
  misc_feature 1..25: note = Single strand DNA oligonucleotide
  source 1..25: mol_type = other DNA
ggcctgtcatatcacatttggcggt

SEQ ID NO: 68 - DNA, 25 bp, synthetic construct
  misc_feature 1..25: note = Single strand DNA oligonucleotide
  source 1..25: mol_type = other DNA
agatgtacaacttatttatgcacag

SEQ ID NO: 69 - DNA, 25 bp, synthetic construct
  misc_feature 1..25: note = Single strand DNA oligonucleotide
  source 1..25: mol_type = other DNA
ggccctgtgcataaataagttgtac

SEQ ID NO: 70 - DNA, 25 bp, synthetic construct
  misc_feature 1..25: note = Single strand DNA oligonucleotide
  source 1..25: mol_type = other DNA
agatttcccaagagtataatgctca

SEQ ID NO: 71 - DNA, 25 bp, synthetic construct
  misc_feature 1..25: note = Single strand DNA oligonucleotide
  source 1..25: mol_type = other DNA
ggcctgagcattatactcttgggaa

SEQ ID NO: 72 - DNA, 25 bp, synthetic construct
  misc_feature 1..25: note = Single strand DNA oligonucleotide
  source 1..25: mol_type = other DNA
agataggaagggaggtttccattgt

SEQ ID NO: 73 - DNA, 25 bp, synthetic construct
  misc_feature 1..25: note = Single strand DNA oligonucleotide
  source 1..25: mol_type = other DNA
ggccacaatggaaacctcccttcct

SEQ ID NO: 74 - DNA, 25 bp, synthetic construct
  misc_feature 1..25: note = Single strand DNA oligonucleotide
  source 1..25: mol_type = other DNA
agatccatactcagcagctggcatt

SEQ ID NO: 75 - DNA, 25 bp, synthetic construct
  misc_feature 1..25: note = Single strand DNA oligonucleotide
  source 1..25: mol_type = other DNA
ggccaatgccagctgctgagtatgg

SEQ ID NO: 76 - DNA, 25 bp, synthetic construct
  misc_feature 1..25: note = Single strand DNA oligonucleotide
  source 1..25: mol_type = other DNA
agattctgataggaaagtaaatcct

SEQ ID NO: 77 - DNA, 25 bp, synthetic construct
  misc_feature 1..25: note = Single strand DNA oligonucleotide
  source 1..25: mol_type = other DNA
ggccaggatttactttcctatcaga

SEQ ID NO: 78 - DNA, 25 bp, synthetic construct
  misc_feature 1..25: note = Single strand DNA oligonucleotide
  source 1..25: mol_type = other DNA
agatgcgctatctatcgcgatgtca

SEQ ID NO: 79 - DNA, 25 bp, synthetic construct
  misc_feature 1..25: note = Single strand DNA oligonucleotide
  source 1..25: mol_type = other DNA
ggcctgacatcgcgatagatagcgc

SEQ ID NO: 80 - DNA, 25 bp, synthetic construct
  misc_feature 1..25: note = Single strand DNA oligonucleotide
  source 1..25: mol_type = other DNA
agatttcaaggcctcgacatgacga

SEQ ID NO: 81 - DNA, 25 bp, synthetic construct
  misc_feature 1..25: note = Single strand DNA oligonucleotide
  source 1..25: mol_type = other DNA
ggcctcgtcatgtcgaggccttgaa

SEQ ID NO: 82-91 - skipped (10 entries)

SEQ ID NO: 92 - DNA, 25 bp, synthetic construct
  misc_feature 1..25: note = Single strand DNA oligonucleotide
  source 1..25: mol_type = other DNA
agatctcacctgatgctgacaagta

SEQ ID NO: 93 - DNA, 25 bp, synthetic construct
  misc_feature 1..25: note = Single strand DNA oligonucleotide
  source 1..25: mol_type = other DNA
ggcctacttgtcagcatcaggtgag

SEQ ID NO: 94 - DNA, 25 bp, synthetic construct
  misc_feature 1..25: note = Single strand DNA oligonucleotide
  source 1..25: mol_type = other DNA
agattcaacttaagagcagaagact

SEQ ID NO: 95 - DNA, 25 bp, synthetic construct
  misc_feature 1..25: note = Single strand DNA oligonucleotide
  source 1..25: mol_type = other DNA
ggccagtcttctgctcttaagttga

SEQ ID NO: 96-97 - skipped (2 entries)

SEQ ID NO: 98 - DNA, 20 bp, synthetic construct
  misc_feature 1..20: note = Single strand DNA oligonucleotide
  source 1..20: mol_type = other DNA
tatgcacaatctcccaagca

SEQ ID NO: 99-114 - skipped (16 entries)

SEQ ID NO: 115 - DNA, 25 bp, synthetic construct
  misc_feature 1..25: note = Single strand DNA oligonucleotide
  source 1..25: mol_type = other DNA
tgctttctacatgtatctttatgac

SEQ ID NO: 116 - DNA, 20 bp, synthetic construct
  misc_feature 1..20: note = Single strand DNA oligonucleotide
  source 1..20: mol_type = other DNA
tttcaccagatttacccatc

SEQ ID NO: 117 - protein, 754 aa, Solanum lycopersicum
MATAQSNSPRVFCIGTADTKFDELRFLSEHVRSSLNSFSNKSSFKVGVTVVDVSTSWKETNSCADFDFVPSKDVLSCHTLGEETMGTFADIRGLAIAIMSKALETFLSIANDEQNLAGVIGLGGSGGTSLLSSAFRSLPIGIPKVIISTVASGQTESYIGTSDLVLFPSVVDICGINNVSKVVLSNAGAAFAGMVIGRLESSKEHSITNGKFTVGVTMFGVTTPCVNAVKERLVKEGYETLVFHATGVGGRAMEDLVRGGFIQGVLDITTTEVADYVVGGVMACDSSRFDAILEKKIPLVLSVGALDMVNFGPKTTIPPEFQQRKIHEHNEQVSLMRTTVGENKKFAAFIAEKLNKASSSVCVCLPEKGVSALDAPGKDFYDPEATSCLTRELQMLLENNERCQVKVLPYHINDAEFANALVDSFLEISPKSRHVECQPAESKSIQDIQNDNAVLEKYPSCNGKNFSRLNDFPNAKPETLQKRTVILQKLKDQISKGKPIIGAGAGTGISAKFEEAGGVDLIVLYNSGRFRMAGRGSLAGLLPFADANAIVLEMANEVLPVVKEVAVLAGVCATDPFRRMDNFLKQLESVGFCGVQNFPTVGLFDGNFRQNLEETGMGYGLEVEMIAAAHRMGLLTTPYAFCPDEAVAMAEAGADIIVAHMGLTTSGSIGAKTAVSLEESVTCVQAIADATHRIYPDAIVLCHGGPISSPEEAAYVLKRTTGVHGFYGASSMERLPVEQAITATVQQYKSISME

SEQ ID NO: 118 - DNA, 2718 bp, Solanum lycopersicum
  source 1..2718: mol_type = genomic DNA
gacttttttatttatttaatattaaatattaatattgctccatcggaataaaaataaaaataaaaatggaacctctccacttgacggttgttgccgcctctctgtccaaccgttgccggtgatctttcatcgtcgagattccagaagctaacaactttctaatcttctcaccattctcacactgagttacactctatcgtccattttgaaatctcgattgtaacacgaattgtatcattttgtttgaggaaaatggcaactgcacagagtaattctcctcgagttttctgtatcggaacagctgatacaaaattcgacgagcttcgtttcctctccgagcatgtgagatccagtcttaacagcttctccaataaatcctcattcaaggtaggagtgacagttgttgatgtctcaaccagctggaaggagacaaatagttgtgctgattttgattttgtaccgagtaaggatgtgctgtcatgccatacactaggggaagaaactatgggcacgtttgcagatattagaggcctagctattgcaatcatgagcaaagctcttgaaactttcctaagtatagctaatgatgaacagaatcttgctggagtaattggccttgggggtagtggaggaacatctctattgtcatctgccttccgatctcttccaattgggatcccaaaagttataatatctacagttgccagtggtcaaactgaatcttatattggaacatcagacttggtattgtttccttcagttgtagatatttgtgggattaacaatgtcagtaaggttgttctatctaatgcgggtgcagcatttgctggaatggtgatcgggaggcttgaaagttcaaaagagcatagcatcactaatggaaagtttacagttggtgtaactatgtttggggttacgactccttgtgttaatgctgtcaaagaaagattagtgaaagaaggatatgagactttggtgttccatgccacgggtgtcgggggcagggccatggaggatcttgttagaggaggttttatacagggtgtgctggatattacgacaactgaggttgcagattacgtagttggaggagtaatggcatgtgatagttcccgatttgatgcaatattagagaagaaaattcctttggttctgagtgtgggagcactggatatggtgaattttggtcctaaaactaccatacctcctgagtttcaacaaagaaagatccatgaacataatgagcaggtttccctaatgcgtactacagtaggtgaaaataagaaatttgctgcatttatagcagaaaagttgaacaaggcatcatcaagtgtatgtgtttgcttgccagagaaaggcgtgtctgcattggatgcacccgggaaagacttttatgatcctgaggcaactagttgtcttacacgtgaactacagatgcttcttgaaaataatgaacgttgtcaggttaaggtcctcccttaccatatcaatgatgcggagtttgcaaatgctttagttgattcattcttggaaatctctccgaaatctagacacgtagaatgtcagccagctgagtccaaatctatccaagacattcagaatgataatgctgttctagagaaatatccctcatgcaacgggaaaaacttttctcgcctgaatgactttccaaatgcaaaaccagaaactttgcagaaaagaactgtgatactgcagaaattgaaagatcaaataagtaagggcaagcctattattggggctggtgctggtacaggtatttctgctaagtttgaggaagctggtggtgtagatttgattgtcttgtacaactcagggcgctttaggatggcaggaaggggatccttagctggtctactgccctttgctgatgcaaatgccattgtacttgagatggccaacgaagtattgcctgtggttaaggaagtggcagttctggctggagtttgtgctactgatcctttccgcaggatggacaacttcctgaagcagttggaatccgttggattctgtggggtgcaaaactttccaactgttggtctgtttgacggtaacttcagacaaaatttggaagagactggaatgggttatggcttggaggttgagatgattgcagcagctcacaggatgggccttttgacaaccccatatgctttctgcccagatgaagcagttgctatggcagaagctggtgccgacatcatagttgctcatatggggcttacaacatctggttcaattggtgcaaaaacagccgtctcattggaggaaagtgtaacttgcgttcaagctattgcagatgctactcataggatatatcctgatgcaattgtgctctgccatggaggccctatatcttcccctgaagaagcagcatatgtactgaagagaaccacaggagttcatggattttatggcgcttcaagcatggaaagactaccagttgagcaagctataactgcaactgtccagcagtacaagtctatttctatggagtgagatgcagtggattttggttctttaaatgtccgaacctatatgtatggtcttcacctctttctttatgtatgatcatgtccagggctcctctgtactcatctagcaacttgaaacactacagcactgctgctgcagtttttatctgtccatatgatatcaatttcaacaagttatatcttttcgtcaaaaaaaaaaaaaaaa

SEQ ID NO: 119 - DNA, 23 bp, synthetic construct
  misc_feature 1..23: note = Single strand DNA oligonucleotide
  source 1..23: mol_type = other DNA
gtcgacatggcaactgcacagag

SEQ ID NO: 120 - DNA, 26 bp, synthetic construct
  misc_feature 1..26: note = Single strand DNA oligonucleotide
  source 1..26: mol_type = other DNA
gcggccgcgtcactccatagaaatag

SEQ ID NO: 121 - DNA, 20 bp, synthetic construct
  misc_feature 1..20: note = Single strand DNA oligonucleotide
  source 1..20: mol_type = other DNA
ccttcgcaagacccttcctc

SEQ ID NO: 122 - DNA, 25 bp, synthetic construct
  misc_feature 1..25: note = Single strand DNA oligonucleotide
  source 1..25: mol_type = other DNA
actgaaggaaacaataccaagtctg

SEQ ID NO: 123 - DNA, 20 bp, synthetic construct
  misc_feature 1..20: note = Single strand DNA oligonucleotide
  source 1..20: mol_type = other DNA
ttcctctccgagcatgtgag

SEQ ID NO: 124 - DNA, 20 bp, synthetic construct
  misc_feature 1..20: note = Single strand DNA oligonucleotide
  source 1..20: mol_type = other DNA
tggaagagatcggaaggcag

SEQ ID NO: 125 - DNA, 2265 bp, Solanum lycopersicum
  source 1..2265: mol_type = genomic DNA
atggcaactgcacagagtaattctcctcgagttttctgtatcggaacagctgatacaaaattcgacgagcttcgtttcctctccgagcatgtgagatccagtcttaacagcttctccaataaatcctcattcaaggtaggagtgacagttgttgatgtctcaaccagctggaaggagacaaatagttgtgctgattttgattttgtaccgagtaaggatgtgctgtcatgccatacactaggggaagaaactatgggcacgtttgcagatattagaggcctagctattgcaatcatgagcaaagctcttgaaactttcctaagtatagctaatgatgaacagaatcttgctggagtaattggccttgggggtagtggaggaacatctctattgtcatctgccttccgatctcttccaattgggatcccaaaagttataatatctacagttgccagtggtcaaactgaatcttatattggaacatcagacttggtattgtttccttcagttgtagatatttgtgggattaacaatgtcagtaaggttgttctatctaatgcgggtgcagcatttgctggaatggtgatcgggaggcttgaaagttcaaaagagcatagcatcactaatggaaagtttacagttggtgtaactatgtttggggttacgactccttgtgttaatgctgtcaaagaaagattagtgaaagaaggatatgagactttggtgttccatgccacgggtgtcgggggcagggccatggaggatcttgttagaggaggttttatacagggtgtgctggatattacgacaactgaggttgcagattacgtagttggaggagtaatggcatgtgatagttcccgatttgatgcaatattagagaagaaaattcctttggttctgagtgtgggagcactggatatggtgaattttggtcctaaaactaccatacctcctgagtttcaacaaagaaagatccatgaacataatgagcaggtttccctaatgcgtactacagtaggtgaaaataagaaatttgctgcatttatagcagaaaagttgaacaaggcatcatcaagtgtatgtgtttgcttgccagagaaaggcgtgtctgcattggatgcacccgggaaagacttttatgatcctgaggcaactagttgtcttacacgtgaactacagatgcttcttgaaaataatgaacgttgtcaggttaaggtcctcccttaccatatcaatgatgcggagtttgcaaatgctttagttgattcattcttggaaatctctccgaaatctagacacgtagaatgtcagccagctgagtccaaatctatccaagacattcagaatgataatgctgttctagagaaatatccctcatgcaacgggaaaaacttttctcgcctgaatgactttccaaatgcaaaaccagaaactttgcagaaaagaactgtgatactgcagaaattgaaagatcaaataagtaagggcaagcctattattggggctggtgctggtacaggtatttctgctaagtttgaggaagctggtggtgtagatttgattgtcttgtacaactcagggcgctttaggatggcaggaaggggatccttagctggtctactgccctttgctgatgcaaatgccattgtacttgagatggccaacgaagtattgcctgtggttaaggaagtggcagttctggctggagtttgtgctactgatcctttccgcaggatggacaacttcctgaagcagttggaatccgttggattctgtggggtgcaaaactttccaactgttggtctgtttgacggtaacttcagacaaaatttggaagagactggaatgggttatggcttggaggttgagatgattgcagcagctcacaggatgggccttttgacaaccccatatgctttctgcccagatgaagcagttgctatggcagaagctggtgccgacatcatagttgctcatatggggcttacaacatctggttcaattggtgcaaaaacagccgtctcattggaggaaagtgtaacttgcgttcaagctattgcagatgctactcataggatatatcctgatgcaattgtgctctgccatggaggccctatatcttcccctgaagaagcagcatatgtactgaagagaaccacaggagttcatggattttatggcgcttcaagcatggaaagactaccagttgagcaagctataactgcaactgtccagcagtacaagtctatttctatggagtga

SEQ ID NO: 126 - protein, 754 aa, Solanum lycopersicum; identical to SEQ ID NO: 117

(19) United States
(12) Patent Application Publication
(10) Pub. No.: US 2024/0298590 A1
(43) Pub. Date: Sep. 12, 2024

(54) ToBRFV-TOLERANT OR RESISTANT PLANTS AND METHODS OF PRODUCING SAME

(71) Applicant: The State of Israel, Ministry of Agriculture & Rural Development, Agricultural Research Organization, Rishon-LeZion (IL)

(72) Inventors: Moshe LAPIDOT, Kiryat Ono (IL); Ilan LEVIN, Mazkeret Batia (IL)

(73) Assignee: The State of Israel, Ministry of Agriculture & Rural Development, Agricultural Research Organization, Rishon-LeZion (IL)

(21) Appl. No.: 18/674,993

(22) Filed: May 27, 2024

Related U.S. Application Data
(63) Continuation of application No. PCT/IL2022/051261, filed on Nov. 27, 2022.
(60) Provisional application No. 63/283,317, filed on Nov. 26, 2021.

(51) Int. Cl. A01H 1/04; A01H 1/00; A01H 6/82

(52) U.S. Cl. CPC A01H 1/045; A01H 1/126; A01H 6/825

(57) ABSTRACT

Methods of producing a tomato plant exhibiting tolerance or resistance to ToBRFV are provided. Also provided are methods of identifying such plants and plants derived thereby as well as processed products comprising same.

### RELATED APPLICATIONS

This application is a Continuation of PCT Patent Application No. PCT/IL2022/051261 having International filing date of Nov. 27, 2022 which claims the benefit of priority under 35 USC § 119(e) of U.S. Provisional Patent Application No. 63/283,317 filed Nov. 26, 2021. The contents of the above applications are all incorporated by reference as if fully set forth herein in their entirety.

### SEQUENCE LISTING STATEMENT

The XML file entitled 100492SequenceListing.xml, created on May 23, 2024, comprising 128,583 bytes, submitted concurrently with the filing of this application is incorporated herein by reference.

### FIELD AND BACKGROUND OF THE INVENTION

The present invention, in some embodiments thereof, relates to ToBRFV-tolerant or resistant plants and methods of producing same.

Viral diseases cause serious damage to plants by significantly reducing their yield and fruit quality. Plant viruses are mostly spread by insects, such as aphids, thrips and whiteflies, and are therefore one of the reasons why in many locations production has shifted from open-field to protected environments (Kang et al., 2005). The worldwide yield losses that can be ascribed to plant viruses are estimated to be more than 30 billion US$ annually (Sastry and Zitter, 2014).

One of the most devastating viruses infecting plants of the Solanaceae family and tomatoes (Solanum Lycopesicum) in particular, are Tobamoviruses. Tobamovirus is a genus in the Virgaviridae family that includes about 35 different virus species (Adams et al., 2009). The two best known viruses of this genus are: Tobacco mosaic virus (TMV) and Tomato mosaic virus (ToMV) (Broadbent, 1976; Lewandowski, 2008). Unlike other viruses transmitted by vectors, tobamoviruses are mechanically transmitted and are considered most persistent in terms of their ability to survive outside plant cells and in dead plant tissues (Caldwell, 1959).

For years, the main way to contain tobamoviruses was through preventative agro-technological means. These means include disinfection of agricultural areas and tools, rotation of seeds, use and replacement of detached soils, use of clean propagation material and the removal of infected plants (Broadbent, 1976). On the other hand, genetic resistance or genetic tolerance, if proven effective, are by far the preferred, economically sound and environmentally friendly way to prevent damage caused by the virus (Kang et al., 2005). A host plant is resistant if it can suppress the multiplication of a virus, and consequently suppress the development of disease symptoms. Resistance can range from very high (up to immunity where no virus accumulates in the host; the plant is in fact a non-host), to moderate, or low. However, even for low resistance, the resistant plant will accumulate less virus than the susceptible host and will express milder disease symptoms. Tolerance is a unique instance where in response to virus infection, the host expresses negligible or mild disease symptoms, but supports normal levels of virus multiplication. Thus, the plant rather than being resistant to the virus, “tolerates” the pathogen and despite its presence expresses mild symptoms and produces a good yield (Cooper and Jones, 1983; Walkey, 1985).

Over the past 80 years, great advances have been made in our understanding of plant resistance against viruses. Approximately half of the known plant virus resistance genes are dominant (Belkhadir, 2004). In the last decade, a large number of crop recessive resistance genes were also identified. These resistances are often achieved through the absence of appropriate host factors required by the virus to complete its replication cycle (Nicaise, 2014). Because plant viruses evolve, and at times acquire the ability to overcome resistance, the development of efficient and durable resistances, able to withstand the genetic plasticity of viruses, still represents a major challenge (Nicaise, 2014).

Two genes, Tm-1 and Tm-2, conferring resistance to ToMV have been introgressed into the cultivated tomatoes. The Tm-1 gene, displaying a semi-dominant inheritance, was originally identified from Solanum habrochites (Pelham, 1966; Watanabe, 1987). This gene maps to the tomato chromosome 2 and encodes a ˜80 kDa protein that physically binds to and functionally inhibits the replication proteins of ToMV (Ishibashi et al., 2007). The Tm-2 resistance gene was discovered in Solanum peruvianum and found to confer a higher level of resistance compared to that displayed by Tm-1. The gene maps to the tomato chromosome 9 and harbors two resistant alleles: Tm-2 and Tm-2² (Pelham, 1966; Young Tanksley, 1989), Tm-2² being more durable than Tm-2 (Fraser, 1990, Lapidot and Levin, 2017). Consequently, Tm-2² is both practically and economically more important because it has been widely exploited as a ToMV resistance source in tomato breeding programs, and was found stable and effective for over 40 years. Both Tm-2 and Tm-2² are dominant and encode a member of the CC-NBS-LRR class of resistance proteins (Lanfermeijer et al., 2003).

Recently, a newly discovered tobamovirus that breaks down Tm-2² resistance was identified and named Tomato brown rugose fruit virus (ToBRFV). The virus was reported in Jordan in 2016 (Salem et al., 2016). A commercial tomato hybrid, grown in greenhouses, showed foliar symptoms at the end of the season accompanied with strong brown rugose symptoms on fruits. The causal agent was found to be transmitted mechanically to test plants that were later found positive to the virus. Following sequence comparisons with other tomato-infecting tobamoviruses, the new virus had the highest nucleotide sequence identity (82.4%) with the Ohio V strain of TMV. ToBRFV was first identified in a tomato greenhouse in southern Israel during 2014 on a number of different commercial tomato hybrids (Luria et al., 2017; Maayan et al., 2018). Tomato plants in this greenhouse, carrying the ToMV-resistance gene Tm-2², displayed disease symptoms that included a heavy mosaic pattern on leaves, narrowing of leaves and yellow spotted fruit, causing heavy losses to fruit yield and quality. Within a short period of time, the new virus spread globally: during 2018 it was identified in tomato plants grown in Mexico, USA, Germany, Italy and the Palestinian authority (Camacho-Beltrán et al., 2019; Ling et al., 2019; Menzel et al., 2019; Panno et al., 2019; Alkowni et al., 2019), and recently in Turkey, China, Greece, Egypt and Spain (Fidan et al., 2019; Yan et al., 2019; Beris et al., 2020; Amer and Mahmoud, 2020; Alfaro-Fernández et al., 2020). This very rapid spread demonstrates that ToBRFV has become a worldwide threat to tomato production.

ToBRFV, found to overcome Tm-2² resistance, demonstrates the genetic plasticity of viruses in their interaction with resistance genes. This exemplifies the need for the continuous development of new, more efficient and durable resistances able to withstand a wider range of virus strains, either alone or in combination with other resistances that were already identified.

Additional background art includes:
WO2018/219941
WO2020/249798
WO2021/110855
Zinger et al. Plants 2021, 10, 179. www(dot)doi(dot)org/10(dot)3390/plants 10010179
Lansor—new commercial tomato variety with ToBRFV resistance bred by Syngenta Vegetable Seeds Company, see: www(dot)zeraim(dot)com/en/news/new-varieties/lansor-new-commercial-tomato-variety-tobrfv-resistance-ir).

### SUMMARY OF THE INVENTION

According to an aspect of some embodiments of the present invention there is provided a method of producing a tomato plant exhibiting tolerance to ToBRFV, comprising:
(a) crossing a tomato plant with another tomato plant comprising a nucleic acid sequence variation which results in tolerance to Tomato Brown Rugose Fruit virus (ToBRFV) wherein the nucleic acid sequence variation is on chromosome 11 and comprising a marker selected from the group consisting of Eco105I_9.42, AciI_9.47 and NcoI_9.53, to create an F1 population;
(b) selfing plants of the F1 population to create an F2 population;
(c) selecting following the crossing or selfing a tomato plant exhibiting tolerance to ToBRFV by marker assisted selection, wherein a marker of the marker assisted selection is selected from the group consisting of Eco105I_9.42, AciI_9.47 and NcoI_9.53.

According to an aspect of some embodiments of the present invention there is provided a method of producing a tomato plant exhibiting tolerance to ToBRFV, comprising:
identifying a tomato plant exhibiting tolerance to ToBRFV by marker assisted selection, wherein a marker of the marker assisted selection is selected from the group consisting of Eco105I_9.42, AciI_9.47 and NcoI_9.53.

According to an aspect of some embodiments of the present invention there is provided a method of producing a tomato plant exhibiting tolerance to ToBRFV, comprising:
(a) crossing a tomato plant with another tomato plant comprising a nucleic acid sequence variation which results in tolerance to Tomato Brown Rugose Fruit virus (ToBRFV) wherein the nucleic acid sequence variation is in a gene on chromosome 11 set forth in Solyc11g018770.3.1, to create an F1 population;
(b) selfing plants of the F1 population to create an F2 population;
(c) selecting following the crossing or selfing a tomato plant exhibiting tolerance to ToBRFV by marker assisted selection, wherein a marker of the marker assisted selection is in a gene set forth in Solyc11g018770.3.1.

According to an aspect of some embodiments of the present invention there is provided a method of producing a tomato plant exhibiting tolerance to ToBRFV, comprising:
identifying a tomato plant exhibiting tolerance to ToBRFV by marker assisted selection, wherein a marker of the marker assisted selection is in a gene set forth in Solyc11g018770.3.1.

According to some embodiments of the invention, the method further comprises at least one of repeating step (b) and backcrossing to the tomato plant.

According to an aspect of some embodiments of the present invention there is provided a method of producing a tomato plant exhibiting tolerance to ToBRFV, the method comprising downregulating expression Solyc11g018770.3.1 in a cell of the tomato plant, thereby conferring tolerance to ToBRFV.

According to an aspect of some embodiments of the present invention there is provided a method of producing a tomato plant exhibiting tolerance to ToBRFV, the method comprising downregulating expression of Solyc11g018770.3.1 in a cell of the tomato plant, wherein the tomato is an open-field tomato, thereby conferring tolerance to ToBRFV.

According to some embodiments of the invention, the downregulating expression is by genome editing.

According to some embodiments of the invention, the downregulating expression is by RNA silencing.

According to some embodiments of the invention, the method comprises regenerating a plant from the cell.

According to some embodiments of the invention, the method comprises growing the plant in an open field.

According to an aspect of some embodiments of the present invention there is provided a method of producing a tomato plant exhibiting resistance to ToBRFV, the method comprising, producing a tomato plant exhibiting tolerance to ToBRFV as described herein, wherein the tomato plant comprises in its genome a Tm-1 resistance gene and/or Tm-2 resistance gene, and/or a QTL2 on chromosome 9.

According to an aspect of some embodiments of the present invention there is provided a tomato plant comprising in its genome a nucleic acid sequence variation in a homozygous form in at least one gene set forth in Solyc11g018770.3.1 resulting in tolerance to Tomato Brown Rugose Fruit virus (TOBRFV).

According to some embodiments of the invention, the nucleic acid sequence variation is non-naturally occurring.

According to some embodiments of the invention, the nucleic acid sequence variation is obtained by genome editing, base-editing or prime-editing techniques, preferably by mutagenesis, by TILLING and/or CRISPR/Cas system.

According to some embodiments of the invention, the plant further comprises in its genome a Tm-1 resistance gene on chromosome 2 and/or a QTL2 on chromosome 9.

According to some embodiments of the invention, the tomato plant is S. lycopersicum.

According to some embodiments of the invention, the ToBRFV virus is the Israeli strain of ToBRFV.

According to some embodiments of the invention, the Tm-1 resistance gene and/or Tm-2 resistance gene is in a heterozygous form.

According to an aspect of some embodiments of the present invention there is provided a tomato plant obtainable by the method as described herein.

According to an aspect of some embodiments of the present invention there is provided a tomato plant exhibiting resistance to ToBRFV obtainable by the method as described herein.

According to an aspect of some embodiments of the present invention there is provided a cell of the plant as described herein.

According to an aspect of some embodiments of the present invention there is provided a Plant part of the plant as described herein.

According to an aspect of some embodiments of the present invention there is provided a seed of the plant as described herein.

According to an aspect of some embodiments of the present invention there is provided a tissue culture of the cell or plant part as described herein.

According to an aspect of some embodiments of the present invention there is provided a method of breeding tomato, the method comprising:
(a) providing the plant as described herein; and
(b) subjecting it to a breeding program.

According to an aspect of some embodiments of the present invention there is provided a method of producing tomato fruits, the method comprising:
(a) growing the plant as described herein to the stage of development of fruits; and
(b) harvesting the fruits.

According to some embodiments of the invention, the growing is in an open filed and the harvesting comprises machine harvesting.

According to an aspect of some embodiments of the present invention there is provided a method of processing tomato comprising:
(a) providing tomato fruit of the plant as described herein;
(b) processing the fruit.

According to an aspect of some embodiments of the present invention there is provided a method for improving the yield of tomato plants in an environment infested by ToBRFV or likely to be infection by ToBRFV or reducing the loss on tomato production in condition of ToBRFV infestation or protecting a field, tunnel, greenhouse or glasshouse of tomato plants from TOBRFV infestation, the method comprising growing tolerant or resistant tomato plants as described herein.

According to an aspect of some embodiments of the present invention there is provided an edible processed product of the plant as described herein.

According to some embodiments of the invention, the processed product is selected from the group consisting of a tomato paste, a ketchup, a tomato sauce a tomato soup, a tomato juice, a tomato powder, a tomato dice, a crushed tomato, a chopped tomato and a tomato concentrate.

According to an aspect of some embodiments of the present invention there is provided a modified gene comprising a null mutation which confers resistance to ToBRFV, wherein the modified gene comprises a nucleic acid sequence having at least 90% identity with respect to the cultivated allele of Solyc11g018770.3.1.

According to an aspect of some embodiments of the present invention there is provided a cultivated tomato plant containing a modified gene as described herein.

According to an aspect of some embodiments of the present invention there is provided a probe or primer pair for identifying a marker for a tomato plant exhibiting tolerance to ToBRFV by marker assisted selection, wherein a marker of the marker assisted selection is in a gene set forth in Solyc11g018770.3.1.

According to an aspect of some embodiments of the present invention there is provided a method for protecting a field, tunnel or glasshouse of tomato plants from ToBRFV infestation, comprising of growing resistant or tolerant tomato plants to ToBRFV as defined as described herein.

According to an aspect of some embodiments of the present invention there is provided a method for increasing the number of harvestable or viable tomato plants in an environment infested by ToBRFV comprising growing tomato plants resistant or tolerant to TORBFV as defined as described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fec.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph of mapping-by-sequencing of the gene controlling tolerance in LA2675 [Each chromosome (Chr) is marked by a different color].

FIG. 2 is a schematic illustration showing fine-tune mapping of the gene controlling tolerance in LA2675 (Map of chromosome 11 and the position of markers used are in the left portion of the figure. The recombinant populations analyzed are in the right portion of the figure. Vertical purple lines show the possible location of the gene in each population separately. The chromosomal region between the red broken horizontal lines is where the gene is located based on superimposing the results obtained from the analysis of all eight populations presented. The association analysis of each population is presented at the bottom of the figure.

FIG. 3 is a schematic illustration showing the four genes that were annotated in the introgression carrying the gene controlling tolerance [Based on the Tomato SL4.0 ITAG4.0 in Jbrowse (www(dot)solgenomics(dot)net/jbrowse_solgenomics/].

FIG. 4 is a graphic presentation showing average relative transcription of ALBINO3-like protein (Solyc11g018740.3.1) in inoculated and non-inoculated tolerant (LA2675) and susceptible (LA3310) genotypes (no statistical difference was found between the two genotypes in all time points tested).

FIG. 5 is a graphic presentation showing average relative transcription of the gene encoding the unknown protein (Solyc11g018750.2.1) in inoculated and non-inoculated tolerant (LA2675) and susceptible (LA3310) genotypes (asterisks denote statistically significant difference (P<0.05) in the average relative expression of the gene between lines).

FIG. 6 is a graphic presentation showing average relative transcription of the gene encoding a polynucleotide adenylyltransferase family protein (Solyc11g018770.3.1) in tolerant (LA2675) and the susceptible (LA3310) lines (the asterisk denotes statistically significant difference (P<0.05) in the average relative expression of the gene between lines).

FIG. 7 shows the sequence of CCA2-1 and its variation is susceptible and tolerant variations according to some embodiments of the invention (SEQ ID NO: 1-4). Sequence of Solyc11g018770.3.1 encoding a CCA tRNA nucleotidyltransferase2 in the tolerant LA2675 line in comparison to a susceptible LA3310 line [Highlighted in yellow and underlined are the start (ATG) and stop (TAG) codons; highlighted in green are the single nucleotide polymorphisms (SNPs) that deviate between the two lines within the underlines codons, three of which are non-synonymous as indicated in the table below the sequence].

FIG. 8 shows Tm-1 involvement in resistance control in a F3 segregating population of LA2675×CC4173. Gray bars—average virus level, blue lines-number of plants tested.

### DESCRIPTION OF SPECIFIC EMBODIMENTS OF THE INVENTION

The present invention, in some embodiments thereof, relates to ToBRFV-tolerant or resistant plants and methods of producing same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Tomato brown rugose fruit virus (ToBRFV) was first identified in Israel during October 2014 in a tomato, Solanum lycopersicum, greenhouse. Tomato plants in this greenhouse, carrying the durable resistance gene against tomato mosaic virus, Tm-2², displayed heavy disease symptoms and losses to fruit yield and quality. These plants were found infected with a tobamovirus, similar to that discovered in Jordan.

Whilst searching for genetic elements that would confer tolerance or resistance to ToBRFV, the present inventors screened genotypes and identified those which are resistant or tolerant to ToBRFV. Specifically, 160 genotypes were screened following inoculation with ToBRFV. The screen resulted in the identification of an unexpectedly high number of genotypes characterized by no foliar symptoms but with high viral titer and therefore regarded as tolerant to the virus. A selected genotype, LA2675, displaying a consistent phenotype of tolerance, was further analyzed. It was found that a single recessive gene, mapped to chromosome 11, controls tolerance in this genotype. This locus displayed a strong association with the tolerance trait, explaining nearly 91% of its variation in segregating populations. Finally, fine-tune mapping of the gene controlling tolerance was carried out using recombinant plants. It was found that the gene is located in a 0.11 Mbp introgression, where four genes were annotated: Solyc11g018740.3.1 (encoding ALBINO3-like protein), Solyc11g018750.2.1 (encoding an unknown protein), Solyc11g018760.1.1 (encoding an unknown protein) and Solyc11g018770.3.1 (encoding a polynucleotide adenylyltransferase family protein, with high homology to CCA tRNA nucleotidyltransferase 2). Gene transcription studies showed that Solyc11g018760.1.1 is not transcribed in both LA2675 and the susceptible genotype and was therefore excluded as a possible gene controlling tolerance. Whereas all remaining three genes displayed nucleotide polymorphisms between LA2675 and susceptible lines in their promoter region, only Solyc11g018770.3.1 was found to carry non-synonymous mutations in its coding region in LA2675.

Finally, in order to validate nucleotidyltransferase 2 (Solyc11g018770.3.1), as the gene controlling tolerance, viral-induced-gene-silencing (VIGS) with TRV was employed and validated the gene as such.

The results were achieved over various backgrounds: LA2675 (“A” examples in the Examples section which follows).

These results provide tools for developing novel tomato varieties which are tolerant or resistant to the virus.

Thus, according to an aspect of the invention there is provided a method of producing a tomato plant exhibiting tolerance to ToBRFV, comprising:
(a) crossing a tomato plant with another tomato plant comprising a nucleic acid sequence variation which results in tolerance to Tomato Brown Rugose Fruit virus (ToBRFV) wherein the nucleic acid sequence variation is on chromosome 11 and comprising a marker selected from the group consisting of Eco105I_9.42, AciI_9.47 and NcoI_9.53 to create an F1 population;
b) selfing plants of said F1 population to create an F2 population;
(c) selecting following the crossing or selfing a tomato plant exhibiting tolerance to ToBRFV by marker assisted selection, wherein a marker of the marker assisted selection is selected from the group consisting of Eco105I_9.42, AciI_9.47 and NcoI_9.53.

According to an alternative or an additional aspect of the invention, there is provided a method of producing a tomato plant exhibiting tolerance to ToBRFV, comprising:

identifying a tomato plant exhibiting tolerance to ToBRFV by marker assisted selection, wherein a marker of said marker assisted selection is selected from the group consisting of Eco105I_9.42, AciI_9.47 and NcoI_9.53.

The term “plant” as used herein encompasses whole plants, a grafted plant, ancestors and progeny of the plants and plant parts, including seeds, shoots, stems, roots, rootstock, scion, and plant cells, tissues and organs. The plant may be in any form including suspension cultures, tissue cultures, embryos, meristematic regions, callus tissue, leaves, gametophytes, sporophytes, pollen, and microspores.

As used herein, the term “tomato” refers to a plant, line or population within the species Solanum lycopersicum (synonyms are Lycopersicon lycopersicum or Lycopersicon esculentum) or formerly known under the genus name of Lycopersicon including but not limited to L. cerasiforrne, L. cheesmanii, L. chilense, L. pennellii, L. chmielewskii, L. esculentum (now S. lycopersicum), L. hirsutum, L. parviborum, L. pennellii, L. peruvianum, L. pimpinellifolium, or S. lycopersicoides. The newly proposed scientific name for L. esculentum is lycopersicum. Similarly, the names of the wild species may be altered. L. pennellii has become S. pennellii, L. hirsutum may become S. habrochaites, L. peruvianum may be split into S. ‘N peruvianum’ and S. ‘Callejon de Hueyles’, S. peruvianum, and S. corneliomuelleri, L. parviflorum may become S. neorickii, L. chmielewskii may become S. chmielewskii, L. chilense may become S. chilense, L. cheesmaniae may become S. cheesmaniae or S. galapagense, and L. pimpinellifolium may become S. pimpinellifolium.

According to a specific embodiment, the tomato is Solanum lycopersicum.

Thus, the tomato plant can be of a cultivated genetic background or a wild tomato genetic background.

According to a specific embodiment, the tomato plant is of a cultivated genetic background.

Generally a cultivated tomato refers to tomato which is suitable for consumption and meets the requirements for commercial cultivation, e.g. typically classified as Solanum lycopersicum. In addition to the tomato plants themselves, and the parts thereof suitable for consumption, such as the fruit, the invention comprises parts or derivatives of the plant suitable for propagation. Examples of parts suitable for propagation are organ tissues, such as leaves, stems, roots, shoots and the like, protoplasts, somatic embryos, anthers, petioles, cells in culture and the like. Derivatives suitable for propagation are for instance seeds. The plants according to the invention can be cultivated or propagated in the conventional manner but also by means of tissue culture techniques from plant parts.

According to a specific embodiment, a cultivated tomato refers to a tomato in which more than 90% of the genome is of Solanum lycopersicum.

The present invention is aimed at using any tomato cultivars, such as of domestic use, fresh market tomatoes or processing tomatoes.

As used herein “open-field tomato” refers to tomato grown by commercial growers in direct seeded, large fields—sprawled for machine harvesting and at times artificial ripening off the vine.

The choice of the variety depends on market demand, regional adaptability, disease resistance and the end use of the product. Exemplary segments for fresh market tomatoes include, but are not limited to, Beef (fruit weight of about 220-400 gr), Standard (fruit weight of about 160-220 gr) and Cluster (uniform fruit weight of about 120-180 gr). Such varieties are available from major seed companies e.g., Grodena, Macarena, Estatio, Zouk, Climbo and Climstar, all available from Syngenta. Other varieties can be proprietary or available from other vendors, including but not limited to, Cherry-micro round cherry (up to 5 gr), mini round cherry (7.5-15 gr), mini plum elongated cherry (10-25 gr). Examples for these varieties are: Creativo (Clause), Batico (Nirit seeds), Shiren (Hazera Genetics). Cocktail round and elongated (25-40 gr): Romanita, Cherry and Cocktail with red, yellow, orange, pink, zebra, chocolate background. Examples include, but are not limited to, Summer sun (Hazera Genetics), Black pearl (Burpec), Tyty (Tomodori). Roma determinate and indeterminate (120-200 gr). Examples for the intermediate marker include, but are not limited to, Lancelot (Vilmorin) and Parsifal (Vilmorin). Pink tomato divided to beef (220-400), standard (160-220) and cluster (120-180). Example: Momotaro type, Cor di bue tomato, (150-350 gr), Pinton (250-300 gr), open field tomato-determinate or semi-determinate (180-400 gr).

Exemplary cultivars of processing tomatoes include, but are not limited to, Roma, SUN 6366, AB 2, Heinz 9780, Heinz 9557, Halley 3155 and Hypeel 303.

There are two major types of tomato growth: determinate and indeterminate, each is contemplated herein. Determinate growth produces “bush” tomatoes and which are bred for compactness. The entire plant stops growing once the terminal fruit ripens, the remainder of the fruit all ripen nearly simultaneously, and then the plant dies. Indeterminate growth produces tomatoes that can grow up to 10 feet in height (so-called “vining” tomatoes) and will only stop growing when killed (e.g. by frost). Their fruits ripen sequentially. In a typical plant, all growth arises from the reiteration of modular sympodial units that each produce three leaves and a multiflowered inflorescence. Most field-grown varieties of tomato, including M82, are determinate plants whose shoots produce an average of six sympodial units, each harboring a single inflorescence, within which leaf number gradually decreases before a precocious termination of growth. In general, determinate tomatoes are suitable for open field production. Semi-determinate and indeterminate “cultivated” varieties are suitable for staked cultivation in the open-field or protected nets and for glasshouse cultivation.

According to an embodiment of the invention the tomato plant is a determinate tomato.

According to an embodiment of the invention the tomato plant is an indeterminate tomato.

According to an embodiment of the invention the tomato plant is a semi-determinate tomato.

According to an embodiment, the tomato is selected from the group consisting of a single fruit per truss, branched tomato and cherry tomato.

As used herein “Tobamovirus Tomato Brown Rugose Fruit virus” abbreviated as TBRFV or ToBRFV is a tobamovirus, which is related to tobacco mosaic virus (TMV) and tomato mosaic virus (ToMV). However, this relatively new virus (known since 2014) can overcome the Tm-2² resistance gene, which means that TMV- and ToMV-resistant varieties will be susceptible to this new virus (ToBRFV). Tobamoviruses are stable outside of their host, and the main routes for transmission of ToBRFV are through propagation material (cuttings and grafts) or mechanical means and contact. For example, the movement of virus on contaminated tools, hands and clothing and plant-to-plant contact. As with other tobamoviruses, ToBRFV can be transmitted via seed. However, it has only been found on the seed coat, not within the seed.

Symptoms include:
Mosaics (chlorotic or pale patches) develop on younger leaves in the head and side shoots;
Leaves may be crumpled (puckered) and deformed; in some cases, leaves may be narrowed;
Brown (necrotic) streaks may develop on stems;
Fruit can develop chlorotic marbling, which can appear similar to infection with Pepino mosaic virus;
Fruit may develop brown wrinkled (rugose) patches.

The term “Resistance” is as defined by the ISF (International Seed Federation) Vegetable and Ornamental Crops Section for describing the reaction of plants to pests or pathogens, and abiotic stresses for the Vegetable Seed Industry. Specifically, by resistance, it is meant the ability of a plant variety to restrict at least to some degree the multiplication of the virus. Symptoms, even if present, are mild as compared to susceptible plants.

The term “Tolerance” is used herein to indicate a phenotype of a plant wherein at least some of the disease-symptoms remain absent upon exposure of said plant to an infective dose of virus, but virus multiplication remains unaffected as in susceptible plants. According to some embodiments, tolerant plants are therefore resistant to symptom expression or are symptomless carriers of the virus.

In case of ToBRFV, by leave tolerance, or foliar tolerance, it is meant the phenotype of a plant wherein the disease symptoms on the leaves remain absent upon exposure of said plant to an infective dose of ToBRFV. Disease symptoms on the fruits may however be at times present on infected plants.

By fruit tolerance, in case of ToBRFV, it is meant the phenotype of a plant wherein the disease symptoms on the fruits remain absent upon exposure of said plant to an infective dose of ToBRFV. Disease symptoms on the leaves may however be present on infected plants.

Symptoms on leaves of ToBRFV infection generally include mosaic, distortion of the leaflets and in many cases also shoestrings like symptoms. Symptoms on fruits of TOBRFV infection generally include typical yellow lesions and deformation of the fruits. In many cases there are also “chocolate spots” on the fruits.

As used herein “Susceptibility” refers to the inability of a plant variety to restrict the growth and development of a specified pest or pathogen; a susceptible plant displays the detrimental symptoms linked to the virus infection, namely the foliar damages and fruit damages in case of ToBRFV infection.

As mentioned, in order to confer tolerance or resistance (dependent on the genetics of the tomato plant) to ToBRFV, the method, according to some embodiments thereof, contemplates, crossing a tomato plant with another tomato plant comprising a nucleic acid sequence variation which results in tolerance to Tomato Brown Rugose Fruit virus (ToBRFV) wherein the nucleic acid sequence variation is on chromosome 11 and comprising a marker selected from the group consisting of Eco105I_9.42, AciI_9.47 and NcoI_9.53 to create an F1 population;

This donor plant or donor parent plant comprising a sequence variation in at least a heterozygous form, but in other more preferred embodiments in a homozygous form, is selected in a breeding program.

According to a specific embodiment, the donor plant is genotype LA2675. As shown in the Examples section which follows, this genotype was crossed to the susceptible S. lycopersicum cv. Moneymaker (LA2706) and to S. lycopersicum cv. Moneymaker carrying the Tm-2² gene (LA3310). The resultant F₁ plants were allowed to self-pollinate to obtain segregating F2 populations.

The nucleic acid sequence variation imparts tolerance to ToBRFV.

As used herein “nucleic acid sequence variation” refers to an allelic variation between susceptible plants and non-susceptible plants.

A tolerant plant is typically homozygous to the variation while a susceptible plant may be heterozygous or nil for the variation.

The term may be interchangeably used with a genetic or molecular marker.

As used herein the phrase “genetic marker” or “molecular marker” refers to a nucleic acid variation which is associated with tolerance or resistance to ToBRFV. The molecular marker can be at least one nucleic acid long or more such as a fragment (sequence) of DNA that is associated with a certain location within the genome and with the trait.

Thus, the variation that may be of a single base, a few bases (2-10), or more (e.g., 11-100) bases to several hundreds e.g., 101-900 or thousands 1 Kb-1000 Kb or 1 Kb-10000 Kb or more of nucleotides conferring the tolerance.

The sequence variation may be selected from the group consisting of a substitution, an insertion, a deletion, a repeat, an inversion and a combination of same.

Methods of validating such variations and their association with the phenotype, in this case “tolerance or resistance to ToBRFV” are well known in the art and described in length in the Examples section which follows (and in WO2020/249798).

As used herein “haplotype” refers to a combination of particular alleles present within a particular plant's genome at two or more linked marker loci, for instance at two or more loci on a particular linkage group.

The genetic marker is in a coding sequence or a non-coding sequence.

To detect nucleic acid sequence variation associated with the tolerance trait, the following criterions are applied: (1) such nucleotides should be in a homozygous state in the tolerant parent and identical in the tolerant F₂ DNA pool, because the tolerance trait was found to be recessive (as detailed in the results); (2) such nucleotides should be polymorphic between the tolerant and the susceptible parents (homozygous nucleotides in the tolerant parent should be replaced by a different nucleotide in a homozygous state in the susceptible parent because the two lines are open-pollinated); and (3) homozygous nucleotides in the tolerant parent should be replaced by a different nucleotide in a homozygous state or be in heterozygous state in the susceptible DNA pool.

These criteria led to the identification of 184,401 SNPs, of which 140,583 were mapped to chromosome 11. Of which, markers mapped to an 0.11 Mb region on chromosome 11 are contemplated herein. These are flanked by markers Eco105I_9.42 and NcoI_9.53

Molecular markers associated with the trait of interest may be identified by one or more methodologies. In some examples one or more markers are used, including but not limited to AFLPs, RFLPs, ASH, SSRs, SNPs, indels, padlock probes, molecular inversion probes, microarrays, sequencing, and the like. In some methods, a target nucleic acid is amplified prior to hybridization with a probe. In other cases, the target nucleic acid is not amplified prior to hybridization, such as methods using molecular inversion probes (see, for example Hardenbol et al. (2003) Nat Biotech 21:673-678). In some examples, the genotype related to a specific trait is monitored, while in other examples, a genome-wide evaluation including but not limited to one or more of marker panels, library screens, association studies, microarrays, gene chips, expression studies, or sequencing such as whole-genome resequencing and genotyping-by-sequencing (GBS) may be used. In some examples, no target-specific probe is needed, for example by using sequencing technologies, including but not limited to next-generation sequencing methods (see, for example, Metzker (2010) Nat Rev Genet. 11:31-46; and, Egan et al. (2012) Am J Bot 99:175-185) such as sequencing by synthesis (e.g., Roche 454 pyrosequencing, Illumina Genome Analyzer, and Ion Torrent PGM or Proton systems), sequencing by ligation (e.g., SOLID from Applied Biosystems, and Polnator system from Azco Biotech), and single molecule sequencing (SMS or third-generation sequencing) which eliminate template amplification (e.g., Helicos system, and PacBio RS system from Pacific BioSciences). Further technologies include optical sequencing systems (e.g., Starlight from Life Technologies), and nanopore sequencing (e.g., GridION from Oxford Nanopore Technologies). Each of these may be coupled with one or more enrichment strategies for organellar or nuclear genomes in order to reduce the complexity of the genome under investigation via PCR, hybridization, restriction enzyme (see, e.g., Elshire et al. (2011) PLOS ONE 6:e19379), and expression methods. In some examples, no reference genome sequence is needed in order to complete the analysis.

Thus, according to some embodiments, determining sequence variation comprises DNA sequencing of said at least one genetic marker.

According to some embodiments, determining sequence variation comprises amplifying said at least one genetic marker.

According to some embodiments, the at least one marker is detected by at least one primer pair or probe. Such primer pairs are provided in Tables 4 below, which should be considered as an integral part of the instant specification.

Thus, in some embodiments, the molecular markers or marker loci are detected using a suitable amplification-based detection method. In these types of methods, nucleic acid primers are typically hybridized to the conserved regions flanking the polymorphic marker region. In certain methods, nucleic acid probes that bind to the amplified region are also employed. In general, synthetic methods for making oligonucleotides, including primers and probes, are well known in the art. For example, oligonucleotides can be synthesized chemically according to the solid phase phosphoramidite triester method described by Beaucage and Caruthers (1981) Tetrahedron Letts 22:1859-1862, e.g., using a commercially available automated synthesizer, e.g., as described in Needham-VanDevanter, et al. (1984) Nucleic Acids Res. 12:6159-6168. Oligonucleotides, including modified oligonucleotides, can also be ordered from a variety of commercial sources known to persons of skill in the art.

It will be appreciated that suitable primers and probes to be used can be designed using any suitable method. It is not intended that the invention be limited to any particular primer, primer pair or probe. For example, primers can be designed using any suitable software program, such as LASERGENE® or Primer3®.

According to some embodiments, the primer pair is selected from the list of Table 1 below which should be considered as an integral part of the instant specification.

It is not intended that the primers be limited to generating an amplicon of any particular size. For example, the primers used to amplify the marker loci and alleles herein are not limited to amplifying the entire region of the relevant locus. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

PCR, RT-PCR, and LCR are in particularly broad use as amplification and amplification-detection methods for amplifying nucleic acids of interest (e.g., those comprising marker loci), facilitating detection of the markers. Details regarding the use of these and other amplification methods are well known in the art and can be found in any of a variety of standard texts. Details for these techniques can also be found in numerous references, such as Mullis, et al. (1987) U.S. Pat. No. 4,683,202; Arnheim & Levinson (1990) C&EN 36-47; Kwoh, et al. (1989) Proc. Natl. Acad. Sci. USA 86:1173; Guatelli, et al., (1990) Proc. Natl. Acad. Sci. USA87:1874; Lomell, et al., (1989) J. Clin. Chem. 35:1826; Landegren, et al., (1988) Science 241:1077-1080; Van Brunt, (1990) Biotechnology 8:291-294; Wu and Wallace, (1989) Gene 4:560; Barringer, et al., (1990) Gene 89:117, and Sooknanan and Malek, (1995) Biotechnology 13:563-564.

Such nucleic acid amplification techniques can be applied to amplify and/or detect nucleic acids of interest, such as nucleic acids comprising marker loci. Amplification primers for amplifying useful marker loci and suitable probes to detect useful marker loci or to genotype SNP alleles are provided. However, one of skill will immediately recognize that other primer and probe sequences could also be used. For instance primers to either side of the given primers can be used in place of the given primers, so long as the primers can amplify a region that includes the allele to be detected, as can primers and probes directed to other SNP marker loci. Further, it will be appreciated that the precise probe to be used for detection can vary, e.g., any probe that can identify the region of a marker amplicon to be detected can be substituted for those examples provided herein. Further, the configuration of the amplification primers and detection probes can, of course, vary. Thus, the compositions and methods are not limited to the primers and probes specifically recited herein.

### Examples of Detection Primers:

Sl11g018770-F5TGCTTTCTACATGTATCTTTATGAC (SEQ ID NO: 179);

Sl11g018770-R5TTTCACCAGATTTACCCATC (SEQ ID NO: 180).

In certain examples, probes will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. A probe can also constitute radiolabelled PCR primers that are used to generate a radiolabelled amplicon. Strategies for labeling nucleic acids and corresponding detection strategies can be found, e.g., in Haugland (1996) Handbook of Fluorescent Probes and Research Chemicals Sixth Edition by Molecular Probes, Inc. (Eugene Oreg.); or Haugland (2001) Handbook of Fluorescent Probes and Research Chemicals Eighth Edition by Molecular Probes, Inc. (Eugene Oreg.).

Detectable labels may also include reporter-quencher pairs, such as are employed in Molecular Beacon and TaqMan™ probes. The reporter may be a fluorescent organic dye modified with a suitable linking group for attachment to the oligonucleotide, such as to the terminal 3′ carbon or terminal 5′ carbon. The quencher may also be an organic dye, which may or may not be fluorescent, depending on the embodiment. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively.

Selection of appropriate reporter-quencher pairs for particular probes may be undertaken in accordance with known techniques. Fluorescent and dark quenchers and their relevant optical properties from which exemplary reporter-quencher pairs may be selected are listed and described, for example, in Berlman, Handbook of Fluorescence Spectra of Aromatic Molecules, 2nd ed., Academic Press, New York, 1971, the content of which is incorporated herein by reference. Examples of modifying reporters and quenchers for covalent attachment via common reactive groups that can be added to an oligonucleotide in the present invention may be found, for example, in Haugland, Handbook of Fluorescent Probes and Research Chemicals, Molecular Probes of Eugene, Oreg., 1992, the content of which is incorporated herein by reference.

In certain examples, reporter-quencher pairs are selected from xanthene dyes including fluorescein and rhodamine dyes. Many suitable forms of these compounds are available commercially with substituents on the phenyl groups, which can be used as the site for bonding or as the bonding functionality for attachment to an oligonucleotide. Another useful group of fluorescent compounds for use as reporters are the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like. In certain other examples, the reporters and quenchers are selected from fluorescein and rhodamine dyes. These dyes and appropriate linking methodologies for attachment to oligonucleotides are well known in the art.

Suitable examples of reporters may be selected from dyes such as SYBR green, 5-carboxyfluorescein (5-FAM™ available from Applied Biosystems of Foster City, Calif.), 6-carboxyfluorescein (6-FAM), tetrachloro-6-carboxyfluorescein (TET), 2,7-dimethoxy-4,5-dichloro-6-carboxyfluorescein, hexachloro-6-carboxyfluorescein (HEX), 6-carboxy-2′,4,7,7′-tetrachlorofluorescein (6-TET™ available from Applied Biosystems), carboxy-X-rhodaminc (ROX), 6-carboxy-4′,5′-dichloro-2′,7′-dimethoxyfluorescein (6-JOE™ available from Applied Biosystems), VIC™ dye products available from Molecular Probes, Inc., NED™ dye products available from Applied Biosystems, and the like. Suitable examples of quenchers may be selected from 6-carboxy-tetramethylrhodamine, 4-(4-dimethylaminophenylazo) benzoic acid (DABYL), tetramethylrhodaminc (TAMRA), BHQ-0™, BHQ-1™, BHQ-2™, and BHQ-3™, each of which are available from Biosearch Technologies, Inc. of Novato, Calif., QSY-7™, QSY-9™, QSY-21™ and QSY-35™, each of which are available from Molecular Probes, Inc., and the like.

In one aspect, real time PCR or LCR is performed on the amplification mixtures described herein, e.g., using molecular beacons or TaqMan™ probes. A molecular beacon (MB) is an oligonucleotide which, under appropriate hybridization conditions, self-hybridizes to form a stem and loop structure. The MB has a label and a quencher at the termini of the oligonucleotide; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, such as to a region of an amplicon during amplification), the MB label is unquenched. Details regarding standard methods of making and using MBs are well established in the literature and MBs are available from a number of commercial reagent sources. See also, e.g., Leone, et al. (1995) Nucl Acids Res. 26:2150-2155; Tyagi and Kramer (1996) Nat Biotechnol 14:303-308; Blok and Kramer (1997) Mol Cell Probes 11:187-194; Hsuih. et al. (1997) J Clin Microbiol 34:501-507; Kostrikis et al. (1998) Science 279:1228-1229; Sokol, et al. (1998) Proc. Natl. Acad. Sci. USA 95:11538-11543; Tyagi, et al. (1998) Nat Biotechnol 16:49-53; Bonnet, et al. (1999) Proc. Natl. Acad. Sci. USA 96:6171-6176; Fang, et al. (1999) J. Am. Chem. Soc. 121:2921-2922; Marras, et al. (1999) Genet. Anal. Biomol. Eng. 14:151-156; and Vet, et al. (1999) Proc. Natl. Acad. Sci. USA 96:6394-6399. Additional details regarding MB construction and use is found in the patent literature, e.g., U.S. Pat. Nos. 5,925,517; 6,150,097; and 6,037,130.

Another real-time detection method is the 5′-exonuclease detection method, also called the TaqMan™ assay, as set forth in U.S. Pat. Nos. 5,804,375; 5,538,848; 5,487,972; and 5,210,015, each of which is hereby incorporated by reference in its entirety. In the TaqMan™ assay, a modified probe, typically 10-25 nucleic acids in length, is employed during PCR which binds intermediate to or between the two members of the amplification primer pair. The modified probe possesses a reporter and a quencher and is designed to generate a detectable signal to indicate that it has hybridized with the target nucleic acid sequence during PCR. As long as both the reporter and the quencher are on the probe, the quencher stops the reporter from emitting a detectable signal. However, as the polymerase extends the primer during amplification, the intrinsic 5′-to-3′ nuclease activity of the polymerase degrades the probe, separating the reporter from the quencher, and enabling the detectable signal to be emitted. Generally, the amount of detectable signal generated during the amplification cycle is proportional to the amount of product generated in each cycle.

It is well known that the efficiency of quenching is a strong function of the proximity of the reporter and the quencher, i.e., as the two molecules get closer, the quenching efficiency increases. As quenching is strongly dependent on the physical proximity of the reporter and quencher, the reporter and the quencher are preferably attached to the probe within a few nucleotides of one another, usually within 30 nucleotides of one another, more preferably with a separation of from about 6 to 16 nucleotides. Typically, this separation is achieved by attaching one member of a reporter-quencher pair to the 5′ end of the probe and the other member to a nucleotide about 6 to 16 nucleotides away, in some cases at the 3′ end of the probe.

Separate detection probes can also be omitted in amplification/detection methods, e.g., by performing a real-time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

Further, it will be appreciated that amplification is not a requirement for marker detection—for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established and are taught, e.g., in Sambrook, et al., Molecular Cloning-A Laboratory Manual (3d ed.), Vol. 1-3, Cold Spring Harbor Laboratory, Cold Spring Harbor, N.Y., 2000 (“Sambrook”); Current Protocols in Molecular Biology, F. M. Ausubel, et al., eds., Current Protocols, a joint venture between Greene Publishing Associates, Inc. and John Wiley & Sons, Inc., (supplemented through 2002) (“Ausubel”)) and PCR Protocols A Guide to Methods and Applications (Innis, et al., eds) Academic Press Inc. San Diego, Calif. (1990) (Innis). Additional details regarding detection of nucleic acids in plants can also be found, e.g., in Plant Molecular Biology (1993) Croy (ed.) BIOS Scientific Publishers, Inc.

Other techniques for detecting SNPs can also be employed, such as allele specific hybridization (ASH). ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

Real-time amplification assays, including MB or TaqMan™ based assays, are especially useful for detecting SNP alleles. In such cases, probes are typically designed to bind to the amplicon region that includes the SNP locus, with one allele-specific probe being designed for each possible SNP allele. For instance, if there are two known SNP alleles for a particular SNP locus, “A” or “C,” then one probe is designed with an “A” at the SNP position, while a separate probe is designed with a “C” at the SNP position. While the probes are typically identical to one another other than at the SNP position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain SNP locus is 11-20 nucleotides in length, dual-labeled with a florescence quencher at the 3′ end and cither the 6-FAM (6-carboxyfluorescein) or VIC (4,7,2′-trichloro-7′-phenyl-6-carboxyfluorescein) fluorophore at the 5′ end.

To effectuate SNP allele detection, a real-time PCR reaction can be performed using primers that amplify the region including the SNP locus, for instance the sequences listed in Table 1, the reaction being performed in the presence of all allele-specific probes for the given SNP locus. By then detecting signal for each detectable label employed and determining which detectable label(s) demonstrated an increased signal, a determination can be made of which allele-specific probe(s) bound to the amplicon and, thus, which SNP allele(s) the amplicon possessed. For instance, when 6-FAM- and VIC-labeled probes are employed, the distinct emission wavelengths of 6-FAM (518 nm) and VIC (554 nm) can be captured. A sample that is homozygous for one allele will have fluorescence from only the respective 6-FAM or VIC fluorophore, while a sample that is heterozygous at the analyzed locus will have both 6-FAM and VIC fluorescence.

The KASPar® and Illumina® Detection Systems are additional examples of commercially-available marker detection systems. KASPar® is a homogeneous fluorescent genotyping system which utilizes allele specific hybridization and a unique form of allele specific PCR (primer extension) in order to identify genetic markers (e.g. a particular SNP locus associated with chloride salt stress tolerance). Illumina® detection systems utilize similar technology in a fixed platform format. The fixed platform utilizes a physical plate that can be created with up to 384 markers. The Illumina® system is created with a single set of markers that cannot be changed and utilizes dyes to indicate marker detection.

These systems and methods represent a wide variety of available detection methods which can be utilized to detect markers associated with tolerance, but any other suitable method could also be used.

According to an embodiment, the introgression is bordered by Eco105I_9.42, and NcoI_9.53

According to an alternative or an additional aspect of the invention, there is provided a method of producing a tomato plant exhibiting tolerance to ToBRFV, comprising:

Hence, a tomato variety is crossed with another plant comprising the variation to arrive at an F1 population, which is typically of hybrid plants.

This population is subjected to selfing.

As used herein “selfing” refers to the fusion of male and female gametes from a single genetic individual. or population.

The steps of selfing, crossing and selection may be repeated as needed (e.g., 2, 3, 4, 5 or more times).

After each step of crossing or selfing a step of selection of tomato plants exhibiting the tolerance can be made. Preferably this is achieved by marker assisted selection (MAS), where the marker is selected from the group of Eco105I_9.42, AciI_9.47 and NcoI_9.53.

According to a specific embodiment, the marker is flanked by Eco105I_9.42 and NcoI_9.53.

As mentioned, according to a specific embodiment, the nucleic acid sequence variation can be in a coding region, non-coding region or an intergenic region.

An example of a gene in which mutations are associated with tolerance to ToBRFV is Solyc11g018770.3.1.

As used herein “Solyc11g018770.3.1” refers to Solanum lycopersicum”, “Enzyme classification.EC_2 transferases.EC_2.7 transferase transferring phosphorus-containing group (50.2.7:264.4)”, “protein_coding”.

This gene has high homology to CCA tRNA nucleotidyltransferase 2.

The gene has two splicing variants CCA2-1 and CCA2-2. SEQ ID NO: 1 corresponds to an exemplary embodiment of a nucleic acid sequence of a tolerance conferring gene; SEQ ID NO: 2 corresponds to an exemplary embodiment of an amino acid sequence of a tolerance conferring gene; SEQ ID NO: 3 corresponds to an exemplary embodiment of a nucleic acid sequence of a susceptible gene; SEQ ID NO: 4 corresponds to an exemplary embodiment of an amino acid sequence of a susceptible gene.

According to a specific embodiment, the modification which confers tolerance is only in CCA2-1. CCA2-2 is not in the introgression sight.

Each of these tolerance conferring genes comprising at least one sequence alteration which confers the tolerance alone or combined (2, 3, 4, 5, 6 or 7) is contemplated to confer tolerance.

It will be appreciated that under a specific embodiment, the mutation in Solyc11g018770.3.1. i.e., CCA2-1 (SEQ ID NO: 1-2) is specifically contemplated to impart tolerance in open-field tomatoes.

Hence, tomatoes of the invention which have tolerance, can be obtained in a more directed manner, e.g., by down-regulating expression of Solyc11g018770.3.1. VIGS assay validated Solyc11g018770.3.1. i.e., CCA1 as at least a single gene that can confer tolerance. In a directed manner it is contemplated down regulating expression of this gene while leaving other genetic sequences not on this locus or these aforementioned loci of chromosome 11 unmodified.

Thus, according to an aspect of the invention there is provided method of producing a tomato plant exhibiting tolerance to ToBRFV, the method comprising downregulating expression of Solyc11g018770.3.1 in a cell of the tomato plant, wherein the tomato is an open-field tomato, thereby conferring tolerance to ToBRFV.

As used herein the phrase “dowregulates expression” refers to dowregulating the expression of a protein (e.g. SOLYC11G018770.3.1) at the genomic (e.g. homologous recombination and site specific endonucleases) and/or the transcript level using a variety of molecules which interfere with transcription and/or translation (e.g., RNA silencing agents) or on the protein level (e.g., aptamers, small molecules and inhibitory peptides and the like).

Down regulation of expression may be either transient or stable.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction as compared to that in the tomato plant in which down-regulation has not been performed, yet of the same genetic background and developmental stage.

Non-limiting examples of agents capable of down regulating SOLYC11G018770.3.1 expression are described in details hereinbelow.

### Down-Regulation at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

According to specific embodiments, the downregulating agent is a polynucleotide.

According to specific embodiments, the downregulating agent is a polynucleotide capable of hybridizing to a gene or mRNA encoding the protein e.g., Solyc11g018770.3.1.

According to specific embodiments, the downregulating agent directly interacts with the gene e.g., Solyc11g018770.3.1.

According to specific embodiments, the agent directly binds the gene e.g., Solyc11g018770.3.1.

According to specific embodiments the downregulating agent is an RNA silencing agent or a genome editing agent.

Thus, downregulation of gene expression e.g., Solyc11g018770.3.1 can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., Solyc11g018770.3.1 and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Downregulation of Solyc11g018770.3.1 can also be achieved by inactivating the gene via introducing targeted mutations involving loss-of function alterations (e.g. point mutations, deletions and insertions) in the gene structure.

Thus, there is provided a modified gene comprising a loss of function mutation e.g., null mutation which confers resistance to ToBRFV, wherein said modified gene comprises a nucleic acid sequence having at least 90% identity with respect to SEQ ID NO: 13.

As used herein “a null mutation” or a “null allele” is a mutation that leads to a non-transcribable RNA and/or non-translatable protein product or a protein product which is non-functional. Such as K460E, N545D, R563S and K589N in the amino acid sequence of Solyc11g018770.3.1 or in the nucleotide sequence such as in the CDS in positions 1620, 1676, 1753 and 282).

Also provided is a cultivated tomato plant containing the modified gene as described herein.

As used herein, the phrase “loss-of-function alterations” refers to any mutation in the DNA sequence of a gene (e.g., Solyc11g018770.3.1) which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to a specific embodiment, the mutation is a null mutation.

As used herein “a null mutation” is a gene mutation that leads to its not being transcribed into RNA and/or translated into a functional protein. According to a specific embodiment, the mutation causes the protein not being translated at all or completely degraded (e.g., as determined by Western blot).

According to a specific embodiment, the loss of function mutation is in the coding sequence of the gene (i.e., not regulatory sequence), such as in the first exon, for instance of Solyc11g018770.3.1).

Examples include, but are not limited to:

Sl11g018770-103rev-
(SEQ ID NO: 156)
FAGATCTCACCTGATGCTGACAAGTA
Sl11g018770-103rev
(SEQ ID NO: 157)
RGGCCTACTTGTCAGCATCAGGTGAG
Sl11g018770-65For-
(SEQ ID NO: 158)
FAGATTCAACTTAAGAGCAGAAGACT
Sl11g018770-65For-
(SEQ ID NO: 159)
RGGCCAGTCTTCTGCTCTTAAGTTGA

According to a specific embodiment, the gene/plant which exhibits tolerance to the ToBRFV comprises a nucleic acid sequence variation which is non-naturally occurring.

According to specific embodiments, loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the e.g. Solyc11g018770.3.1 may be in a homozygous form. According to this embodiment, homozygosity is a condition where both alleles at the e.g. Solyc11g018770.3.1 locus are characterized by the same nucleotide sequence. Heterozygosity refers to different mutations in the gene at the e.g. Solyc11g018770.3.1 locus.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENS—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): c82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

CRISPR-Cas system-Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinck et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of a gRNA that can be used in the present invention are shown in B Examples of the Examples section which follows. According to a specific embodiment, the introduced variation mimics the naturally occurring variation.

According to another specific embodiment, the introduced variation confers a non-naturally occurring variation.

In order to use the CRISPR system, both gRNA and Cas9 should be expressed in a target cell. Cas9 can also be provided as mRNA or protein to the cell. The insertion vector can contain both cassettes on a single plasmid or the cassettes are expressed from two separate plasmids. CRISPR plasmids are commercially available such as the px330 plasmid from Addgene.

“Hit and run” or “in-out”-involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy-involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologous targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the PI bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Constructs useful in the methods according to the present invention, such as for down-regulating expression of Solyc11g018770.3.1 may be constructed using recombinant DNA technology well known to persons skilled in the art. The coding sequence constructs may be inserted into vectors, which may be commercially available, suitable for transforming into plants and suitable for expression of the gene of interest in the transformed cells. The genetic construct can be an expression vector wherein the nucleic acid sequence is operably linked to one or more regulatory sequences allowing expression in the plant cells.

Plant cells may be transformed stably or transiently with the nucleic acid constructs or with naked DNA or RNA of the present invention. In stable transformation, the nucleic acid molecule of the present invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217;

Glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

However other methods of production are also contemplated including sexual reproduction (and selection for the phenotype whether morphologically or using molecular markers as described herein), tissue culture and more.

Micropropagation is a process of growing new generation plants from a single piece of tissue that has been excised from a selected parent plant or cultivar. This process permits the mass reproduction of plants having the preferred tissue expressing the fusion protein. The new generated plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-frec. During stage two, the initial tissue culture is multiplied until a sufficient number of tissue samples are produced to meet gradually increased so that it can be grown in the natural environment.

Viruses that have been shown to be useful for the transformation of plant hosts include CaMV, TMV, TRV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

It will be appreciated that nucleic acid variations may be obtained by the use of mutagens which may be combined with regeneration or additional breeding as needed. Examples of chemical mutagens include, but are not limited to nitrous acid, alkylating agents such as ethyl methanesulfonate (EMS), methyl methane sulfonate (MMS), diethylsulfate (DES), and base analogs such as 5-bromo-deoxyuridine (5BU). Physical mutagens include radiation (e.g. fast neutron, gamma radiation).

Following transformation and optionally regeneration, the plant material (e.g., cell, seed, plantlet etc.) can be used as is or subjected to further breeding.

Thus, once a plant carrying the nucleic acid sequence variation preferably in a homozygous form is identified it is considered as being tolerant to ToBRFV. This plant material can be used as a breeding material in the development of a tomato plant varieties having agriculturally desired traits.

It will be appreciated that in order to improve tolerance or resistance, the plant comprising the nucleic acid sequence variation on chromosome 11 can be pre-selected comprising QTLs which are associated with tolerance on chromosome 9, i.e., QTL2, and optionally express Tm-1 and/or Tm-2 resistance genes, as previously described in WO2020/249798 (Tables 3 and 4 therein), which is hereby incorporated by reference in its entirety.

The Tm-1 gene is as defined inter alia in the publication Ishibashi et al., 2007 (An inhibitor of viral RNA replication is encoded by a plant resistance gene. PNAS Aug. 21, 2007 104 (34) 13833-13838); preferably ‘Tm-1 gene’ refers to a genetic sequence encoding a protein having the Tm-1 activity reported in the article, namely the ability to inhibit the viral replication of a wild-type ToMV strain Tm-1 sensitive, for example the strain ToMV-L disclosed in this article. According to a preferred embodiment, the Tm-1 gene according to the invention is a gene encoding a protein having the 754 amino acid sequence reported in Ishibashi et al, corresponding to NCBI BAF75724, or a protein having at least 75%, preferably at least 80%, more preferably at least 85%, 90%, or 95% sequence identity NCBI BAF75724 and exhibiting the Tm-1 activity reported in Ishibashi et al, 2007, namely the ability to inhibit viral RNA replication of a wild-type Tm-1 sensitive ToMV strain. According to a preferred embodiment, this gene has a sequence corresponding to the mRNA sequence referred to in Ishibashi et al, 2007, namely sequence NCIB AB287296, or a sequence having at least 50%, preferably at least 60%, at least 70%, more preferably at least 75%, 80%, 85%, 90%, or 95% sequence identity with NCIB AB287296. Irrespective of the degree of sequence identity with NCIB, a Tm-1 gene according to the invention preferably encodes a protein exhibiting the Tm-1 activity reported in Ishibashi et al, 2007, namely the ability to inhibit viral RNA replication of wild-type ToMV.

It is preferred that, in the genome of a plant, seed or cell of the invention, the Tm-1 gene be present on chromosome 2. The present invention however also encompasses plant, seed or cell, comprising the Tm-1 gene at a locus which does not correspond to the locus mentioned in Ishibashi et al, 2007.

According to a specific embodiment, Tm-2 is used along with the herein described sequence variations to obtain ToMV and ToBRFV resistance in a single genotype.

According to one embodiment, the plants of the present invention are of a hybrid variety—i.e. are generated following the crossing (i.e. mating) of two non-isogenic plants both being homozygous for a loss of function mutation in the mentioned markers on chromosome 11 or the Solyc11g018770.3.1. The hybrid may be an F₁ Hybrid.

An “F₁ Hybrid” as used herein, refers to first generation progeny of the cross of two non-isogenic plants.

The development of tomato hybrids of the present invention requires the development of stable parental lines. In breeding programs desirable traits from two or more germplasm sources or gene pools are combined to develop superior breeding varieties. Desirable inbred or parent lines are developed by continuous self-pollinations and/or backcrosses and selection of the best breeding lines, sometimes utilizing molecular markers to speed up the selection process (e.g., Eco105I_9.42, AciI_9.47 and NcoI_9.53).

Once the parental lines that give the best hybrid performance have been identified e.g., both carrying the loss of function mutation as described above e.g., in the Solyc11g018770.3.1 gene or markers Eco105I_9.42, AciI_9.47 and NcoI_9.53, the hybrid seed can be produced indefinitely, as long as the homozygosity of the parents are maintained. According to one embodiment the tomato plants of the present invention are stable parent plant lines (e.g., carrying the loss of function mutation e.g., in the Solyc11g018770.3.1 gene in a heterozygous form or a homozygous form).

As defined herein, the phrase “stable parental lines” refers to open pollinated, inbred lines, stable for the desired plants over cycles of self-pollination and planting. According to a specific embodiment, 95% of the genome is in a homozygous form in the parental lines of the present invention.

A common practice in plant breeding is using the method of backcrossing to develop new varieties by single trait conversion.

The phrase “single trait conversion” as used herein refers to the incorporation of new single gene into a parent line wherein essentially all of the desired morphological and physiological characteristics of the parent lines are recovered in addition to the single gene transferred.

The term “backcrossing” as used herein refers to the repeated crossing of a hybrid progeny back to one of the parental tomato plant. The parental tomato plant which contributes the gene for the desired characteristic is termed the non-recurrent or donor parent, as mentioned hereinabove. This terminology refers to the fact that the non-recurrent parent is used one time in the backcross protocol and therefore does not recur. The parental plant to which the gene from the non-recurrent parent are transferred is known as the recurrent parent as it is used for several rounds in the backcrossing protocol.

In a typical backcross protocol, a plant from the original varieties of interest (recurrent parent) is crossed to a plant selected from second varieties (non-recurrent parent) that carries the gene, introgression or hamplotype of interest to be transferred. The resulting progeny from this cross are then crossed again to the recurrent parent and the process is repeated until a tomato plant is obtained wherein essentially all of the desired morphological and physiological characteristics of the recurrent parent are recovered in the converted plant, in addition to the transferred gene from the non-recurrent parent.

Thus, near-isogenic lines (NIL) may be created by many backcrosses to produce an array of individuals that are nearly identical in genetic composition except for the trait or genomic region under interrogation in this case loss of function genetic alteration e.g., in the Solyc11g018770.3.1 gene.

Backcrossing methods can be used with the present invention to improve or introduce a characteristic into the parent lines. Marker assisted breeding (selection) as described above can be used in this method.

According to a specific embodiment, the plant or the plant seed is an inbred.

According to a specific embodiment, the plant is a hybrid plant or the seed is a hybrid seed.

The invention also relates to progeny of the tomato of the invention. Such progeny can be produced by sexual or vegetative reproduction of a plant of the invention or a progeny plant thereof. In addition to this, the progeny plant may be modified in one or more other characteristics. Such additional modifications are for example effected by mutagenesis or by transformation with a transgene.

As used herein the word “progeny” is intended to mean the offspring or the first and all further descendants from a cross with a plant of the invention that shows tolerance to the ToBRFV as described herein. Progeny of the invention are descendants of any cross with a plant of the invention that carries the mutation (in a homozygous form) trait that leads to tolerance. Progeny also encompasses plants that carry the trait of the invention which are obtained from other plants of the invention by vegetative propagation or multiplication.

As mentioned, embodiments described herein, furthermore, relate to hybrid seed and to a method of producing hybrid seed comprising crossing a first parent plant with a second parent plant and harvesting the resultant hybrid seed. In this case the trait is recessive, therefore both parent plants need to be homozygous for the trait in order for all of the hybrid seed to carry the trait of the invention. They need not necessarily be uniform for other traits.

Embodiments described herein also relate to the germplasm of the plants. The germplasm is constituted by all inherited characteristics of an organism and according to the invention encompasses at least the trait of the invention.

Embodiments described herein also relate to cells of the plants that have the tolerance associated markers (Eco105I_9.42, AciI_9.47 and NcoI_9.53 or loss of function mutation in the Solyc11g018770.3.1) and optionally QTL2 and Tm-1 and/or Tm-2 resistance genes. Each cell of such plants carries the genetic information that leads to the trait (i.e., tolerance to ToBRFV). The cell may be an individual cell or be part of a plant or plant part, such as the fruit or seed.

Thus, is some embodiments of the invention, there is provided a tomato plant comprising in its genome a nucleic acid sequence variation in a homozygous form in at least one gene selected from the group consisting of optionally Solyc11g018770.3.1 resulting in tolerance to Tomato Brown Rugose Fruit virus (TOBRFV).

According to an embodiment of the invention, the plant has been genetically edited to produce said nucleic acid sequence variation and may or may not comprise coding region for the gene editing agent e.g., Cas9.

According to an embodiment, the plant further comprises in its genome any of a Tm-1 or Tm-2 resistance genes, and/or a QTL2 on chromosome 9.

According to a specific embodiment, the Tm-1 resistance gene and/or the Tm-2 resistance gene are in a heterozygous form.

As mentioned, the newly identified locus of Solyc11g018770.3.1 confers tolerance of ToBRFV. However, adding any of QTL2 and/or Tm-1 resistance genes may confer resistance to ToBRFV.

Hence, there is provided a tomato plant exhibiting tolerance or resistance to ToBRFV obtainable by the methods described herein.

Also provided a cell, plant part (e.g., seed) or tissue culture of such plants.

By increasing tolerance to biotic stress, tomato yield may be increased especially in an environment infested or likely to be infected with ToBRFV.

The invention is thus also directed to a method for improving the yield of tomato plants in an environment infested, or likely to be infected by ToBRFV, e.g., the Israeli strain or isolate, comprising growing TOBRFV-resistant tomato plants according to the invention [that have the tolerance associated markers (Eco105I_9.42, AciI_9.47 and NcoI_9.53 or loss of function mutation in the Solyc11g018770.3.1 gene) optionally QTL2 and Tm-1 resistance gene]. Preferably QTL2 is present homozygously. According to another embodiment, at least one is present heterozygously, preferably with another one present homozygously. Preferably, the method comprises a first step of choosing or selecting a tomato plant having at least one of the tolerance QTLs and the Tm-1 gene. The method can also be defined as a method of increasing the productivity of a tomato field, tunnel, greenhouse or glasshouse.

As used herein “yield” refers to the number and/or weight of harvested tomato fruits per plant or per cultivated area.

As disclosed in the preceding aspect, the tomato plant to be grown preferably also comprises a Tm-2 or Tm-2² allele, preferably heterozygously.

These methods are particularly valuable for a population of tomato plants, either in a field, in tunnels, greenhouses or in glasshouses.

Alternatively, said methods for improving the yield or reducing the loss on tomato production may comprise a first step of identifying tomato plants resistant to ToBRFV and comprising in their genome a tolerance QTL on chromosome 11 (as defined by the markers of Eco105I_9.42, AciI_9.47 and NcoI_9.53) or loss of function in the mentioned locus on said chromosome homozygously in combination with a Tm-1 gene and optionally QTL2, and then growing said resistant plants in an environment infested or likely to be infested by the virus.

The resistant plants of the invention are also able to restrict and even inhibits the growth of ToBRFV, especially the Israeli isolate or strain of ToBRFV, thus limiting the infection of further plants and the propagation of the virus. Accordingly, the invention is also directed to a method of protecting a field, tunnel, greenhouse or glasshouse, or any other type of plantation, from ToBRFV infestation, or of at least limiting the level of infestation by ToBRFV of said field, tunnel, greenhouse or glasshouse or of limiting the spread of ToBRFV in a field, tunnel, greenhouse or glasshouse, especially in a tomato field. Such a method preferably comprises the step of growing a resistant plant of the invention [that have the tolerance associated markers (Eco105I_9.42, AciI_9.47 and NcoI_9.53 or loss of function mutation in the Solyc11g018770.3.1 gene) and Tm-1 resistance genes and optionally QTL2]

Thus, there is also provided a method for protecting a field, tunnel or glasshouse of tomato plants from ToBRFV infestation, comprising of growing resistant or tolerant tomato plants to as described herein.

Alternatively or additionally, there is provided a method for increasing the number of harvestable or viable tomato plants in an environment infested by ToBRFV comprising growing tomato plants resistant or tolerant to ToRBFV as described herein.

The methods may also comprise a subsequent step of harvesting tomatoes.

According to a specific embodiment, the growing is in an open-field and the harvesting is by machine harvesting.

The present teachings further relate to consumed products which comprise the genomic (DNA) information that leads to the above-mentioned tolerance or resistance to ToBRFV and therefore tomatoes which are endowed with this genetic background are devoid of aberrations characteristic for these types of infections.

Thus, there is provided a method of processing tomato comprising:
(a) providing tomato fruit of the tolerant or resistanct plant, as described herein; and
(b) processing said fruit.

As such embodiments of the invention, also relate to an edible processed product of the plant as described herein.

Fruits of any of the plants described herein may be selected or qualified for fruit color, Brix, pH, sugars, organic acids and defect levels (insect damage, mold, etc.) at ripening or post-harvest. For example, tomatoes are typically transported to a large processing facility, where they are collected and where they may subsequently be washed, typically using chlorinated water and rinsed using tap water and further selected to remove those that present defects (e.g., inadequate ripening, disease damage, molds etc.). Tomatoes may be stored (especially those exhibiting improved shelf-life as described above) or immediately sent to the consumer (fresh-market tomatoes). Processing tomatoes may be processed into a wide variety of products.

For juice or pulp production, the tomatoes may be subject to oven dehydration and are comminuted and macerated (disintegrated and broken) to obtain a pumpable mass. As will be clear to the skilled person these operations per se are known and common in the field of tomato processing and any adjustments to the method can be made in this regard without departing from the scope.

Methods for processing tomatoes and/or producing tomato-based compositions are well known in the art, see generally U.S. Pat. No. 6,924,420. Also reported are specific methods for preparing, for example, paste (U.S. Pat. No. 7,074,451), sterile paste (U.S. Pat. No. 4,206,239), puree (U.S. Pat. No. 4,556,576), sauce (U.S. Pat. No. 7,122,217), solidified sauce (U.S. Pat. No. 4,038,424), barbecue sauce (U.S. Pat. No. 6,869,634), salsa (U.S. Pat. No. 5,914,146), ketchup (U.S. Pat. No. 6,689,279), tomato fiber composition (U.S. Pat. No. 7,166,315) and dehydrated tomato-product (U.S. Pat. No. 5,035,909). Methods of modifying the texture and consistency of tomato paste, pulp, and puree has also been reported, see, for example, U.S. Pat. No. 6,720,019.

Also provided is an edible processed tomato product comprising the tomato or an edible portion thereof (e.g., fruit or an edible part thereof).

Also provided is a tomato paste generated according to the present teachings.

Examples of such edible products include, but are not limited to, canned tomatoes (whole), a tomato paste, a ketchup, a tomato sauce a tomato soup, a dehydrated tomato, a tomato juice, a tomato powder, a tomato dice, a crushed tomato, a chopped tomato and a tomato concentrate.

According to some embodiments, the products comprise the DNA carrying markers of Eco105I_9.42, AciI_9.47 and NcoI_9.53 or loss of function mutation in the Solyc11g018770.3.1 and optionally QTL2 and/or Tm-1 resistance genes.

It will be appreciated that the genes described herein may confer tolerance or resistance to any tobamovirus not limited to ToBRFV.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Plant Material and Resource Populations

Seeds of tomato genotypes were obtained from the Tomato Genetics Resource Center (TGRC) at the University of California, Davis and from seed stocks available at the Volcani Center, Israel. These genotypes included cultivated commercial hybrids or their selections in advanced generations, wild tomato species, in particular those closely related to the cultivated tomato; genotypes displaying amino-acid variation at the Tm-2 locus (based on the genomic browser available at www(dot)solgenomics(dot)net/jbrowse_solgenomics); and genotypes carrying the Tm-1, Tm-2 and Tm-2² genes, either alone or in combination. At least eight plants of 160 genotypes were inoculated with ToBRFV and eight of these genotypes were separately inoculated with ToMV.

The tolerant genotype LA2675 was used for further studies. This genotype was crossed to the susceptible S. lycopersicum cv. Moneymaker (LA2706) and to S. lycopersicum cv. Moneymaker carrying the Tm-2² gene (LA3310). The resultant F₁ plants were allowed to self-pollinate to obtain segregating F2 populations.

### Virus Maintenance, Virus Acquisition and Plant Inoculation

ToBRFV (GeneBank Acc. No. KXG619418) was maintained on Moneymaker tomato plants carrying the Tm-2² (LA3310) while ToMV was maintained on Moneymaker (LA2706) plants in an insect-proof greenhouse. The cultures were propagated and renewed every three-to-four weeks by mechanical inoculation. The virus was transmitted mechanically: leaves of ToBRFV-infected tomato source plants were ground in 0.01 phosphate buffer (pH 7.0) and applied to carborundum dusted test plants. The carborundum was washed out and the test plants were kept in a temperature-controlled greenhouse (18/25° C. Min/Max) under natural conditions without artificial light. All inoculations were carried out in 8-row×16-column sowing trays with 40 ml planting soil (Hishtil Plant-Nursery Company, Israel). Inoculations were carried out at the first true leaf stage of the seedlings, approximately two weeks after sowing. Seedlings were fertilized on a weekly basis throughout the experiment. ToBRFV-induced symptoms and viral levels were evaluated using the procedures described below.

### Disease Severity Scoring

ToBRFV- or ToMV-induced symptoms were evaluated 30 days post inoculation (DPI), and at times later, in a temperature controlled greenhouse. The symptoms were evaluated according to the disease severity index (DSI): (0) no visible symptoms, inoculated plants show the same growth and development as non-inoculated plants; (1) light mosaic pattern on the apical leaf; (2) severe mosaic pattern on the apical leaf, (3) very severe mosaic pattern, coupled with pronounced elongation or folding of the apical leaf.

### Enzyme-Linked Immunosorbent Assay (ELISA) to Evaluate Viral Levels

Indirect ELISA analyses were performed on plant leaves using laboratory-produced specific antibodies against ToMV or ToBRFV as previously described (Koenig, 1981; Dombrovsky et al., 2013). In the analysis, two discs, 1 cm in diameter, taken from the 4^{th} and the 5^{th} true leaf represented each plant. Samples were taken 30 days after inoculation, ground in coating buffer (Agdia) and incubated for 3 h at 37° C. with 1:5000 dilution of the specific antiserum (anti-ToBRFV or anti-ToMV). Detection was carried out by incubating the samples with AP-conjugated goat anti-rabbit (IgG) (Sigma, Steinheim, Germany) for 3 h at 37° C. P-nitro phenyl phosphate (Sigma) substrate was used at a concentration of 0.6 mg/mL. The developing color was recorded by ELISA reader (Thermo Fisher Scientific Multiskan FC) at 405 nm and 620 nm. Optical density (OD) values of a minimum ratio of three times the value of the negative, non-infected, controls were considered positive.

### Genomic DNA Extraction and Analysis of Markers by Polymerase Chain Reaction (PCR)

Genomic DNA was extracted from individual plants according to Fulton et al. (1995). PCR primers were designed using the Primer3 software version 4.0 (www(dot)bioinfo(dot)ut(dot)ec/primer3-0.4.0/). The PCRs were performed in a volume of 15 μl containing 10 ng of template DNA, 10 μmol of each of two primers, 0.2 mM of each dNTP, 2 mM MgCl₂, 1 U of Taq DNA polymerase, and 1×PCR-buffer. The PCRs conditions were: 94° C. 3 min, followed by 35 cycles of 94° C. for 30 s, 58-60° C. for 30 s (depending on primers' characteristics), and 72° C. for 1 min. Final elongation was at 72° C. for 10 min. Amplification products were digested with restriction endonucleases and visualized by electrophoresis in 2% agarose gel. Sequences of the primers used in this study are presented in Table 1.

TABLE 1
Primers used to obtain SCAR DNA markers for
association studies on chromosome 11
(in bold are the markers used in the fine tune
mapping).

| | 5′-to-3′ Forward (F) and Reverse |
|---|---|
| Marker name* | (R) primer sequences/SEQ ID NO: |
| PvuI_7.61 | F-GGGAGAATTGAATGTGGAGGGT/19 |
| | R-GACAGGTTCGTGATTGCAGC/20 |
| DraI_8.13 | F-GCAGATTTAGAGGTCAGATCCTTC/21 |
| | R-CATGCCAGTACCAGAGTTCAATAG/22 |
| EcoRI_8.47 | F-TCACTCTCAAAGCAATTCATAATGT/23 |
| | R-GAGCTTCAGTGGGTCTCAAT/24 |
| TaqI_8.79 | F-ATCCTACCAACTCCGAGAATAGAAC/25 |
| | R-GGTGTGGAATCTAGCAACATAAATC/26 |
| MscI_8.87 | F-CCCATGGGTCTTTTTCTCTTTC/27 |
| | R-AATACATGTTGGGTCCTATGTGTG/28 |
| BstNI_8.97 | F-GGTACCCTCTCAATCTCAAGGTC29 |
| | R-GAATTTACACGCCACCTTCCTC/30 |
| BmgBI_8.98 | F-TTCTTCCATTATCACACCATGGG/31 |
| | R-TTGTTTGCTCACAGGCACAC/32 |
| Bsp119I_9.36 | F-TCGAGAGAGGAGAGGTTATAAGGAC/33 |
| | R-GACGGGGTATTCCTTGGTTATC/34 |
| Eco105I_9.42 | F-AGAACTACTGCCTCGAGTTTCTTC/35 |
| | R-ACTCTCGAGTCTAGACACTCATTGG/36 |
| AciI_9.47 | F-CTCGCGTCCAATGGTTTGAG/37 |
| | R-TGCACCTTCAATTAACGTGAGAC/38 |
| NcoI_9.53 | F-GACTTGAGTAACTGGGAAACTGGAC/39 |
| | R-GGTGCCCAAAGTATGATGAATG/40 |
| DraI_9.66 | F-GAAGAAGAAGCAGGCCAGAAAG/41 |
| | R-GAGATAGCCGAGCGATATTGAG/42 |
| HhaI_9.87 | F-TCCAAGTGGCATGTTTAATGAC/43 |
| | R-CGAGTTCCAATACTTTCCCATC/44 |
| BcuI_10.03 | F-GTCGTGACAGAAATAGATGGAATG/45 |
| | R-TACATCTGACCCTCATATGCTAGG/46 |
| EcoRI_48.57 | F-CAGAGAGTTACACACGACCAAGTC/47 |
| | R-AGGTTATCTTCTTTAACCCCCAAG/48 |

*Marker name is composed of the restriction endonuclease used to digest the PCR products and its map location in Mbp on chromosome 11 based on Tomato SL4.0 ITAG4.0 in Jbrowse (www(dot)solgenomics(dot)net/jbrowse_solgenomics/).

### RNA Extraction and Transcriptional Analysis

Relative transcript levels of genes was determined by HTS of cDNA obtained from inoculated and non-inoculated parental lines, LA2675 and LA3310, in two biological repeats. Initial samples were taken prior to the inoculation from the apex of eight plants of each of the two parental lines at their first true leave stage. These samples represented the start of the experiment (0 DPI). Additional samples were taken in a similar manner from inoculated and non-inoculated parental plants at 5, 10, 15 and 30 DPI.

Total RNA was extracted using TRI-reagent (Sigma-Aldrich, St. Louis, MO) and DNA contaminants were digested with TURBO DNA-free DNAase (Ambion, Austin, TX). cDNA synthesis, libraries preparation and HTS analysis were carried out at the Crown institute for genomics, the Weizmann Institute, Rehovot, Israel.

### Mapping-by-Sequencing of the Gene Controlling Tolerance

To map the gene controlling tolerance the procedure presented by Soyk et al., 2017 was followed. An F2 population was generated by crossing the tolerant LA2675 with the susceptible Moneymaker genotype, carrying the Tm-2² gene (LA3310). From 335 ToBRFV-inoculated F2 plants, 25 plants displaying the most severe symptoms (DSI=3.0), and 25 plants displaying no visible symptoms, but virus infected as confirmed by Elisa were selected. DNA was extracted from each one of these plants using the DNeasy Plant Mini Kit (QIAGEN, www(dot)qiagen(dot)com/us/). 0.4 μg of DNA was taken to represent each plant in the susceptible and the symptomless DNA pool. In addition, six μg of DNA were extracted from each one of the two parental lines. HTS analysis of the four DNA samples, including the libraries preparation, were carried out in Cornell University, Ithaca, New York, USA as described by Soyk et al., 2017 with the following modification: ˜100 bp paired-ends sequencing reads were obtained using the Illumina HighSeq2000 machine.

On average, 125,012,108 (˜40 Gb) reads were obtained. The FASTX Toolkit (www(dot)hannonlab(dot)cshl(dot)edu/fastx_toolkit/) was used for: (1) trimming read-end nucleotides with quality scores <30 using fastq_quality_trimmer; (2) removing reads with a quality score ≤30 using fastq quality filter. ˜91% of an average total of 94,657,458 cleaned reads, obtained after processing and cleaning, were successfully mapped onto the SOL reference genome database available at www(dot)solgenomics(dot)net/, version Sol 3.0, using the Burrows-Wheeler Aligner (BWA) software with its default parameters (Li and Durbin, 2010). The resulting mapping file was processed using SAMtools/Picard tool (www(dot)broadinstitute(dot)github(dot)io/picard, version 1.78; Li et al., 2009) for adding read group information, sorting, marking duplicates and indexing. Then, the local realignment process for locally realigning reads was performed so that the number of mismatching bases is minimized across all the reads using the RealignerTargetCreator and IndelRealigner of the Genome Analysis Toolkit version 3.4-0 (GATK; version www(dot)broadinstitute(dot)org/gatk/; DePristo et al. 2011). Finally, the variant calling procedure was performed using HaplotypeCaller of the GATK toolkit. Only sites with DP (read depth) higher than 20 were analyzed.

To detect nucleotides associated with the tolerance trait, the following criterions were applied: (1) such nucleotides should be in a homozygous state in the tolerant parent and identical in the tolerant F₂ DNA pool, because the tolerance trait was found to be recessive (as detailed in the results); (2) such nucleotides should be polymorphic between the tolerant and the susceptible parents (homozygous nucleotides in the tolerant parent should be replaced by a different nucleotide in a homozygous state in the susceptible parent because the two lines are open-pollinated); and (3) homozygous nucleotides in the tolerant parent should be replaced by a different nucleotide in a homozygous state or be in heterozygous state in the susceptible DNA pool. These criteria led to the identification of 184,401 SNPs, of which 140,583 were mapped to chromosome 11.

### Fine-Tune Mapping of Genes Controlling Tolerance

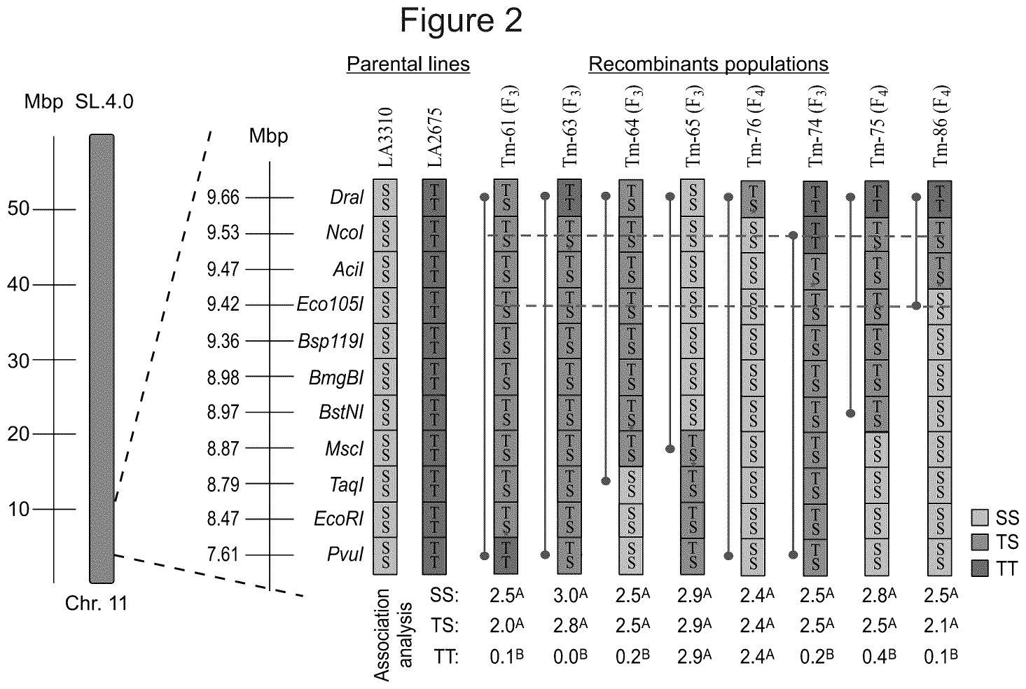

Fine-tune mapping of the gene controlling tolerance was carried out in a similar manner to that described by us earlier (Lapidot et al., 2015). In order to fine-tune map the genes controlling tolerance 2000 F₂ and F₃ plants derived from the initial cross between LA2675 and Moneymaker (LA2706) were genotyped with markers presented in Table 1 to identify recombinant plants (i.e., plants displaying different polymorphisms following PCR analysis with the primers presented in Table 1). Of these plants, the eight most informative recombinant plants were selected for further study: Five F₂ plants and three F₃ plants. These eight plants were allowed to self-pollinate to obtain five F₃ and three F₄ populations. These eight populations represent different introgressions from the tolerant genotype: some of in homozygous state and some in heterozygous state (FIG. 2). A population of 60 plants from each one of these eight populations were inoculated with ToBRFV and their symptoms recorded at 30 DPI. Individual plants of each of these eight populations was genotyped using a different DNA markers expected to segregate in each particular population (Marked by a red asterisks in FIG. 2). Analysis of association between these markers and DSI was performed in each population separately to determine association or disassociation between the marker and DSI in each population separately. For example: The F4 population Tm-86 is fixed for the susceptible alleles for all markers from PvuI_7.52 through Eco105I_9.34, fixed for the tolerant allele at the DraI_9.59 marker and should display segregation in the region marked by AciI_9.39 and NcoI_9.45. Individual plants in this population segregated for DSI levels in agreement with AciI_9.39 and Nco_9.45 markers, demonstrated by the significant association between Nco_9.45 and DSI displayed at the bottom of FIG. 2. Based on this population, the gene controlling tolerance should reside in a 0.25 Mbp region between Eco105I_9.34 and DraI_9.59 as marked by the vertical purple line to its left. Superimposing the results obtained from the eight segregating populations presented in FIG. 2 is therefore expected to yield the location of the gene controlling tolerance as marked by the two horizontal broken lines in FIG. 2.

### Transcriptional Analysis

Raw-reads were subjected to a filtering and cleaning procedure. The SortMeRNA tool was used to filter out rRNA. Next, the FASTX Toolkit (www(dot)hannonlab(dot)cshl(dot)edu/fastx_toolkit/index(dot)html, version 0.0.13.2) was used to trim read-end nucleotides with quality scores <30, using the FASTQ Quality Trimmer, and to remove reads with less than 70% base pairs with a quality score ≤30 using the FASTQ Quality Filter. Clean reads were mapped to the reference genome of Tomato version SL4.0 (www(dot)biorxiv(dot)org/content/10.1101/767764v1) using Tophat2 software (Kim et al., 2013; v. 2.1) with an average mapping rate of 94%. Gene abundance estimation was performed using Cufflinks (Trapnell et al., 2010; v. 2.2) combined with gene annotations from the Sol Genomics Network version ITAG4.0 (www(dot)solgenomics(dot)net/organism/Solanum_lycopersicum/genome). Gene expression values were computed as FPKM (Fragments Per Kilobase of transcript per Million mapped reads). Heatmap visualization and Principal component analysis (PCA) were performed using R Bioconductor (Gentleman et al., 2004). Differential expression analysis was completed using the edgeR package (Robinson et al., 2010) in the R environment. Genes with an adjusted p-value of no more than 0.05 (Benjamini and Hochberg, 1995), were considered differentially expressed. Venn diagrams were generated using the online tool at bioinformaticsDOTpsbDOTugentDOTbe/webtools/Venn/.

Construction of the CCA tRNA Nucleotidyltransferase 2 RNAi Silencing Vector and its Transformation into Tomato Plants

To silence CCA tRNA nucleotidyltransferase 2, a pHannibal vector (Wesley et al., 2001) expressing a sense and an anti-sense fragment of the gene was constructed in two steps. In the first step, a 408 bp fragment of the gene (cDNA coordinates 1 to 408) was amplified by PCR using the forward primer CCA F-XhoI (5′-AGA CTCGAG ATG AAG GCT TTG AGT ATA GC-3′ SEQ ID NO: 49), containing a XhoI restriction site, and the reverse primer CCA R-KpnI (5′-GAC GGTACC TCG TCC ATA CAT GTT GTC AAG-3′ SEQ ID NO: 50), containing a KpnI site. This fragment was cloned into the unique XhoI and KpnI sites present in the sense oriented arm of pHannibal. In the second step, the same 576 bp fragment of the gene was amplified by PCR performed with the forward primer CCA F-XbaI (5′-GAC TCTAGA ATG AAG GCT TTG AGT ATA GC-3′ SEQ ID NO: 51), containing a XbaI site, and the reverse primer CCA R-BamHI (5′-GAC GGATCC TCG TCC ATA CAT GTT GTC AAG-3′ SEQ ID NO: 52), containing a BamHI site. This fragment was cloned into the unique XbaI and BamHI sites present in the anti-sense oriented arm of pHannibal, thus creating pHannibal-CCA. To create a binary vector, pHannibal-CCA was cloned under the cauliflower mosaic virus (35S) promoter and the nitric oxide synthase transcriptional terminator into the NotI site of the pBIN vector. Transformations were carried out on cotyledon cuttings of susceptible Moneymaker tomato plants with Agrobacterium tumefaciens strain GV3101:pMK90 as previously described (Azari et al., 2010).

Modular Cloning Protocol of the CRISPR/Cas9 Vector for the Gene Encoding CCA tRNA Nucleotidyltransferase 2 (Solyc11g018770.3.1) and its Transformation into Tomato Plants

The Golden Gate modular cloning system (Weber et al., 2011) was used to construct the CRISPR/Cas9 vector for the gene encoding encoding a polynucleotide adenylyltransferase family protein, with high homology to CCA IRNA nucleotidyltransferase 2 (Solyc11g018770.3.1). In order to achieve knockout of the gene, four constructs were designed. Each construct contained a sgRNA targeting different site in gene sequence (Table 2). Constructs 1 and 2 were designed to create defined deletions within the gene sequence, while constructs 3 and 4 were designed to create non-synonymous change and/or reading frameshift in the gene coding sequence.

Oligos containing target site sequence were synthesized by Hy labs (Ness ziona, Israel). Each oligo used as forward primer along with a reverse primer in a PCR reaction in order to generate sgRNA. PCR reaction were performed in a volume of 50 μL, containing 10 μL of HF buffer, 4 μL of dNTP's, 1.5 μL of DMSO, 3 μL of pICH86966 (Addgene no. 48075) carrying sgRNA scaffold, 2.5 μL of the forward guide RNA primer, 2.5 μL Reverse guide RNA primer, and 0.5 μL of fusion Taq (New England Biolabs). PCR reaction condition were: 98° C. for 30 sec., followed by 28 cycles of 98° C. for 10 sec., 55° C. for 30 sec., 72° C. for 30 sec. Final elongation was at 72° C. for 10 min. Each PCR product was visualized by electrophoresis in 2% agarose gel followed by DNA purification using NucleoSpin® PCR clean up kit (Macherey-Nagel, Germany).

Assembly of the AtU6::guide RNA expression cassette for each of the sgRNA separately was performed in a single tube reaction, in volume of 20 μL, containing 60 ng of PCR product from previous step, 200 ng of pICSL01009 (Addgene no. 46968) carrying Arabidopsis (Arabidopsis thaliana) U6 promoter, 950 ng of level 1 acceptor plasmid (Table 2), 2 μL of cut smart buffer, 1 μL of BsaI restriction enzyme (New England Biolabs), 1 μL of ATP 25 mM (Thermo Scientific), 1 μL of ligase in a 1:4 ratio (Thermo Scientific). Reaction condition were: 15 cycles of 40° C. for 3 min, 16° C. for 3 min followed by 50° C. for 20 min, 80° C. for 20 min. reaction product were treated with 1 ul of Plasmid-Safe™ ATP Dependent exo-DNase (Lucigen, USA) for 1 hour in 37° C.

Each of the gRNA products were transformed into E. coli and grown on selective medium containing Ampicillin (100 μg\mL) and x-gal for blue/white selection. Plasmids from Positive colonies were extracted using NucleoSpin® plasmid EasyPure (Macherey-Nagel, Germany) and validated by sequencing (Macrogen Europe B.V., Netherlands).

All level 1 expression cassette and the end-linker pICH50866 (Addgene no. 48022) were assembled into level 2 acceptor pAGM4723 (Addgene no. 48015) already carrying NPTII::Cas9, in order to create binary vector containing one expression cassette: NPTII::Cas9::sgRNA1::sgRNA2::sgRNA3::sgRNA4::End linker. Level 2 reaction was preformed in a volume of 20 μL containing: 950 ng of level 2 acceptor, 300 ng from each level 1 acceptor, 3 μL of end-linker plasmid, 2 μL of ligase buffer 10×, 2 μL of BSA 10×, 1 μL of BpiI, 1 μL of BsaI and 1 μL of ligase T4. Reaction conditions were: 37° C. for 20 sec, followed by 40 cycles of (37° C. for 3 min, 16° C. for 3 min), 50° C. for 10 min and 80° C. for 10 min. level 2 reaction product was treated with 1 μL of Plasmid-Safe™ ATPDependent exo-DNase (Lucigen, USA) for 1 hour in 37° C. and transformed into competent E. coli for selective growth with kanamycin (50 μg/mL). Plasmids were extracted from positive colonies and validated by sequencing. Plasmids containing the correct order of the expression cassette were transformed into Agrobacterium tumefaciens strain GV3101:pMK90 and later into cotyledon cuttings of susceptible Moneymaker tomato plants as previously described (Azari et al., 2010).

TABLE 2
Guide RNA sequences (gRNA) and the reverse primer used for the
construction of the CRISPR/Cas9 vector for the gene encoding CCA
tRNA nucleotidyltransferase 2 (Solyc11g018770.3.1).

| gRNA | Gene target site seq/SEQ ID NO: | Level 1 vector |
|---|---|---|
| 1 | GTAAACATAACCTTTAGTGA/53 | pICH47761 (Addgene |
| | | no. 48003) |
| 2 | GAGCTGGACAAAAGGAACAG/54 | pICH47772 (Addgene |
| | | no. 48004) |
| 3 | CAAAACACTTGGAAACAGCG/55 | pICH47781 (Addgene |
| | | no. 48005) |
| 4 | TTACTTGCTTGGGTTCAGCT/56 | pICH47791 (Addgene |
| | | no. 48006) |
| Revers | TGTGGTCTCAAGCGTAATGCCAA | |
| primer | CTTTGTAC/57 | |

### Quantitative and Qualitative Statistical Analyses

Viral level and DSI recorded at 30 DPI, or later, in foliar tissue of each plant were analyzed. All Analyses were carried out with the JMP Pro 15 statistical discovery software (SAS Institute Inc., Cary, NC, USA). The association between DNA markers and DSI were evaluated by analyses of variance as well as by chi-square (χ²). An excellent agreement was found between χ² and the respective analyses of variance; therefore, analyses of variance are presented for DSI throughout this manuscript. Differences among means are presented as different superscript letters that represent statistically significant mean values (P<0.05) based on Tukey-Kramer Honestly Significant Difference (HSD) test (Kramer 1956).

### Example 1

### ToBRFV is Able to Infect Tomato Plants Harboring Genetic Resistance to ToMV

In Israel, ToBRFV was initially identified in commercial ToMV-resistant tomato plants carrying the Tm-2² gene. To verify that ToBRFV can indeed infect ToMV-resistant plants, tomato open-pollinated genotypes, carrying either the Tm-2 gene, the Tm-2² gene or a combination of Tm-1 and Tm-2² were inoculated. The plants were inoculated with either ToMV or with ToBRFV and compared with an open-pollinated genotype carrying no ToMV-resistance gene. Results presented in Table 3, show that ToMV-resistant genotypes indeed displayed no disease following inoculation with ToMV. However, when infected with ToBRFV, these genotypes displayed very high average disease severity index (DSI) and viral levels, very much like the ToMV-susceptible control.

To test whether Tm-1 is effective against ToBRFV, different Tomato Genetics Resource Center (TGRC) accessions carrying either Tm-1, Tm-2, a combination of Tm-1 and Tm-2 or no ToMV resistance gene were inoculated. Results presented in Table 4 show that the average DSI levels of genotypes carrying Tm-1, either alone or in combination with Tm-2, displayed very high average DSI as did accessions with no ToMV resistance gene and accessions carrying Tm-2 alone.

TABLE 3
Average disease severity index (DSI) and average viral level in ToMV-resistant
and susceptible tomato plants infected with ToMV or with ToBRFV.

| S. lycopersicum | ToMV | | DSI | | Viral level (OD) | |
|---|---|---|---|---|---|---|
| cv. | resistance gene | N | ToMV | ToBRFV | ToMV | ToBRFV |
| Moneymaker | — | 8 | 2.6^{A} ± 0.1 | 2.7^{A} ± 0.1 | 647^{A} ± 10 | 802^{A} ± 25 |
| (LA2706) | | | | | | |
| T-5 (LA2399) | Tm-2/Tm-2 | 8 | 0.3^{B} ± 0.0 | 2.8^{A} ± 0.1 | 11^{B} ± 1 | 731^{A} ± 18 |
| Momor (LA2828) | Tm2²/Tm-2² | 8 | 0.0^{B} ± 0.0 | 3.0^{A} ± 0.0 | 6^{B} ± 1 | 396^{B} ± 5 |
| Vendor (LA2968) | Tm2²/Tm-2² | 8 | 0.0^{B} ± 0.0 | 2.9^{A} ± 0.1 | 5^{B} ± 1 | 733^{A} ± 22 |
| Moneymaker | Tm2²/Tm-2² | 8 | 0.0^{B} ± 0.0 | 3.0^{A} ± 0.0 | 20^{B} ± 2 | 1072^{A} ± 30 |
| (LA3310) | | | | | | |
| Mocimor (LA2830) | Tm-1/Tm-1 and | 8 | 0.4^{B} ± 0.0 | 2.4^{A} ± 0.2 | 6^{B} ± 1 | 659^{A} ± 19 |
| | Tm2²/Tm-2² | | | | | |

Results are presented as Mean ± Standard Error, viral level was determined by ELISA using specific antibodies for each tested virus and is presented as optical-density (OD) × 1000, N denotes number of plants, and different superscript letters above means express a statistically significant difference, P < 0.05, based on Tukey-Kramer Honestly Significant Difference (HSD) test.

TABLE 4
Average disease severity index (DSI) of different TGRC accessions
carrying Tm-1, Tm-2, a combination of Tm-1 and Tm-2 or no
ToMV resistance gene following inoculation with ToBRFV.

| Accession | Genotype | N | DSI |
|---|---|---|---|
| LA2706 | cv. Moneymaker | 8 | 3.0^{A} ± 0.0 |
| LA2838A | cv. Ailsa Craig | 8 | 3.0^{A} ± 0.0 |
| LA2825 | Tm-1/Tm-1 in cv. Moneymaker background | 8 | 3.0^{A} ± 0.0 |
| LA3269 | Tm-1/Tm-1 cv. Ailsa Craig background | 8 | 3.0^{A} ± 0.0 |
| LA3271 | Tm-1/Tm-1 in cv. Ailsa Craig background | 8 | 3.0^{A} ± 0.0 |
| LA3276 | Tm-1/Tm-1 in cv. Ailsa Craig background | 8 | 3.0^{A} ± 0.0 |
| LA3268 | Tm-2/Tm-2 in cv. Craig background | 8 | 3.0^{A} ± 0.0 |
| LA2399 | Tm-2/Tm-2 in cv. T5 background | 8 | 3.0^{A} ± 0.0 |
| LA3297 | Tm-1/Tm-1 and Tm-2/Tm-2 in cv. Vagabond | 8 | 3.0^{A} ± 0.0 |
| | background | | |
| LA3432 | Tm-1/Tm-1, Tm-2/Tm-2 in cv. Ailsa Craig | 8 | 3.0^{A} ± 0.0 |
| | background | | |
| LA3812 | Tm-1/Tm-1, Tm-2/Tm-2 in cv. Ailsa Craig | 8 | 3.0^{A} ± 0.0 |
| | background | | |

Results are presented as Mean ± Standard Error, N denotes number of plants, and identical superscript letters above means express no statistical significant difference, P > 0.05, based on Tukey-Kramer Honestly Significant Difference (HSD) test

### Example 2

### Screening for ToBRFV-Tolerant Genotypes

At least eight plants of each one of 160 genotypes were initially tested for foliar symptoms following inoculation with ToBRFV (total number of plants >1280). At least eight plants of each genotype showing no foliar symptoms were inoculated again to validate their phenotype. Of the 160 genotypes screened, 29 (18.1%) were found tolerant to ToBRFV. Plants of these tolerant genotypes showed no symptoms following inoculation with ToBRFV but were characterized with viral levels that were as high as susceptible genotypes. Of these 29 tolerant genotypes, nine (31.0%) belong to S. pimpinellifolium and eight (27.6%) were cultivated lines or hybrids.

A representative tolerant genotype (LA2675; average DSI=0.1±0.0, average viral level=724±52) was selected for further studies because it displayed the most consistent phenotype following several inoculations.

### Example 3

### Genetic Inheritance of ToBRFV-Tolerance in LA2675

A total of 104 F2 plants of the initial cross between the tolerant LA2675 genotype and a susceptible genotype (S. lycopersicum cv. Moneymaker, LA2706) were inoculated with ToBRFV together with the parental lines and their F₁ hybrid plants to evaluate DSI. Results, presented in Table 5, show that while the tolerant line LA2675 displayed very low average symptom levels, its F₁ crossbred plants with the susceptible line displayed very high average symptom levels that did not differ from the susceptible line. This indicates that the tolerance trait is controlled in a recessive manner. Of the 104 F2 plants inoculated, 25 (24%) showed no symptoms, similarly to the tolerant parent, indicating that a single recessive gene controls tolerance [χ²=0.05, P(χ²)=0.8].

TABLE 5
Average ToBRFV disease severity index (DSI) of the
tolerant genotype LA2675, the susceptible genotype (Moneymaker),
their F₁ hybrid and F₂ plants.

| Genotype | N | DSI |
|---|---|---|
| Moneymaker (LA2706) | 8 | 3.0^{A} ± 0.0 |
| LA2675 | 8 | 3.0^{A} ± 0.2 |
| F₁ (LA2706 × LA2675) | 8 | 3.0^{A} ± 0.0 |
| F₂ (LA2706 × LA2675) | 104 | 2.2^{A} ± 0.1 |

N denotes number of plants, results are presented as Mean ± Standard Error, and different superscript letters above means express a statistical significant difference, P < 0.05, based on Tukey-Kramer Honestly Significant Difference (HSD) test.

### Example 4

### Mapping-by-Sequencing of the Locus Controlling Tolerance

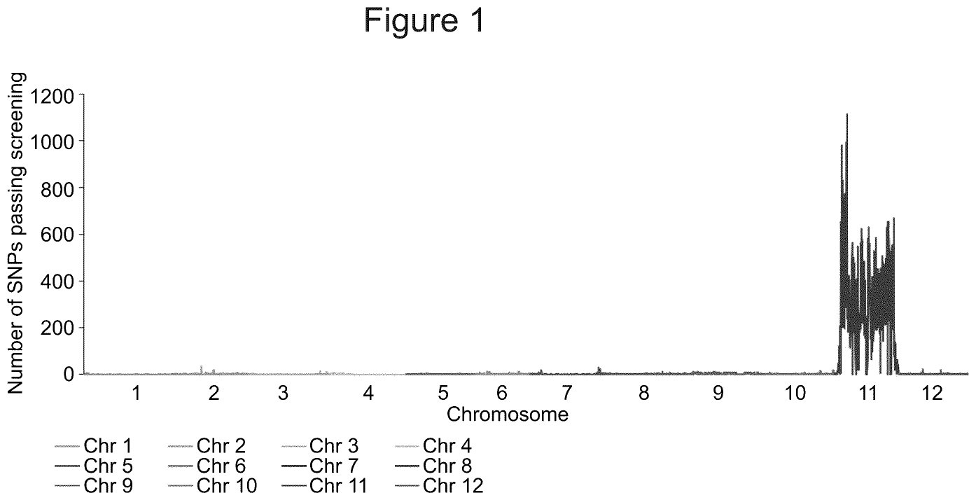

In an effort to identify and map the quantitative trait locus (QTL) controlling tolerance, DNA pools extracted from susceptible and tolerant F2 plants, resulting from a cross between LA2675 and a susceptible Moneymaker genotype, carrying the Tm-2² gene (LA3310, Table 3), as well as DNA samples extracted from their two parental lines were subjected to High Throughput Sequence (HTS) analysis. The HTS analysis results led to the identification of 184,401 single nucleotide polymorphisms (SNPs) passing the screening procedure detailed in the Materials and Methods. Of these 184,401 SNPs, 140,583 (76.2%) were mapped to chromosome 11, while the rest were scattered throughout the entire genome. These results, presented in FIG. 1, point only to chromosome 11 as the one carrying the QTL controlling tolerance.

### Example 5

### Development DNA Markers for the Analysis of Tolerance in LA2675

A set of 15 sequence-characterized amplified region (SCAR) DNA markers, scattered throughout chromosome 11, were developed for the analysis of association with the tolerance trait in LA2675 and recombinant plants (Table 6). Initial analyses of segregating populations, resulting from initial crosses between LA2675 and ToBRFV-susceptible genotypes, revealed that although all of these markers were significantly associated with the tolerance, the BstNI_8.97 marker was one of the markers initially exhibiting the highest level of association and therefore used in the analysis presented in Table 7.

### Example 6

### Analysis of Association Between the BstNI_8.97 Marker and Tolerance in LA2675

An F₂ population of 168 plants resulting from a cross between LA2675 and the susceptible genotype Moneymaker (LA2706) were inoculated with ToBRFV, together with their respective control lines, and their symptoms recorded. The F₂ plants were genotyped with BstNI_8.97 and a highly significant association between this marker and DSI was obtained [P(F)=1.5λ10⁻⁸⁵, R²=91%, Table 7].

TABLE 7
Analysis of association between the BstNI_8.97 marker and ToBRFV
disease severity index (DSI) in the tolerant LA2675 genotype.

| Parental lines and F₁ plants | | | Analysis of association | | |
|---|---|---|---|---|---|
| Genotype | N | DSI | BstNI_8.97 | N | DSI |
| Moneymaker (LA2706) | 8 | 0.0 ± 3.0^{A} | SS | 44 | 2.9^{A} ± 0.0 |
| LA2675 | 8 | 0.2 ± 0.0^{B} | ST | 67 | 2.6^{A} ± 0.1 |
| F₁ (LA2706 × LA2675) | 8 | 0.1 ± 2.9^{A} | TT | 57 | 0.0^{B} ± 0.0 |

The parental lines and their F1 crossbred plants are presented in left portion of the table, while the analysis of association is presented in its right part; BstNI_8.97 genotypes: SS represent homozygous plants with the marker variant inherited from the susceptible parent, TT represent homozygous plants with the marker variant inherited from the tolerant genotype, and ST represent heterozygous plants. N denotes number of plants, results are presented as Mean±Standard Error, and different superscript letters above means express a statistically significant difference, P<0.05, based on Tukey-Kramer Honestly Significant Difference (HSD) test

### Example 7

### Fine Tune Mapping of the Gene Controlling Tolerance in LA2675

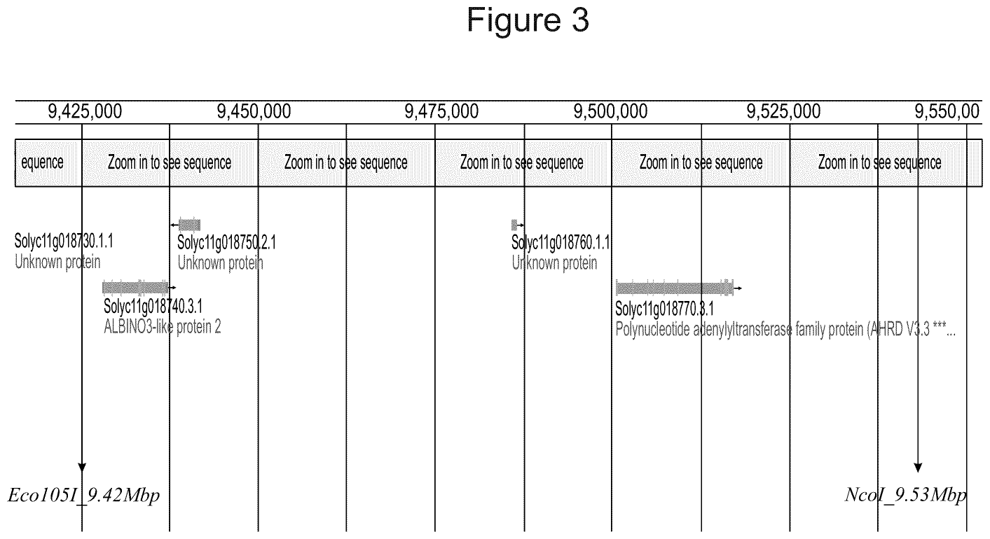

Results presented in FIG. 2 point to a 0.11 Mbp region between Eco105I_9.42 and NcoI_9.53 as the chromosomal region carrying the gene controlling tolerance. In this region four genes were observed: Solyc11g018740.3.1, Solyc11g018750.2.1, Solyc11g018760.1 and Solyc11g018770.3.1 (Tomato SL4.0 ITAG4.0 in Jbrowse (www(dot)solgenomics(dot)net/jbrowse_solgenomics/, FIG. 3). Whereas all of these four genes were found characterized by nucleotide polymorphisms at their promoter region (1,500 bp upstream to their start codon) in the tolerant genotype vs. susceptible genotypes, only Solyc11g018770.3.1 carried non synonymous mutations in its coding region in LA2675, leading to amino acid differences between the susceptible and the tolerant genotype.

### Example 8

### Transcriptional Analysis of the Tolerant and Susceptible Parental Lines

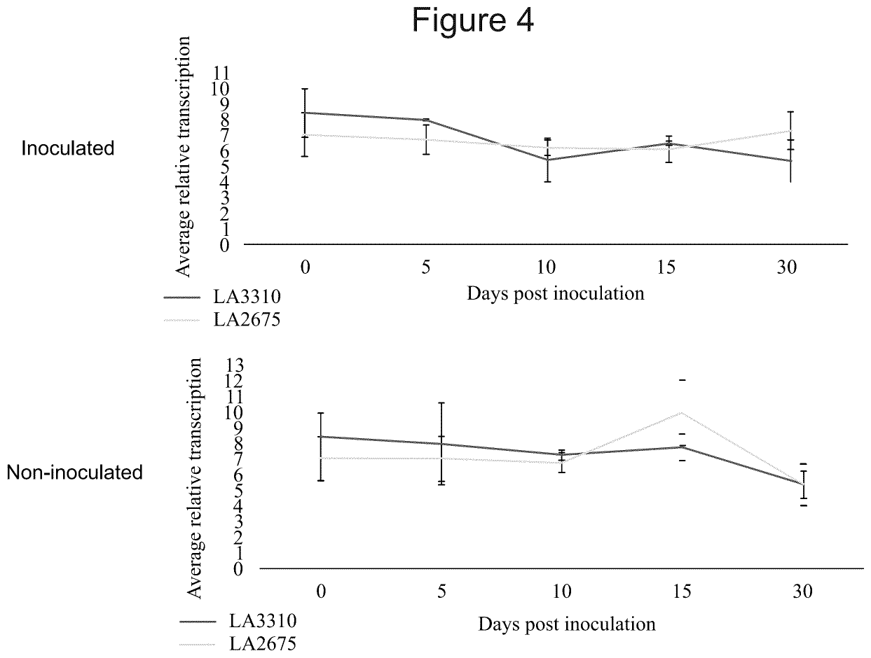

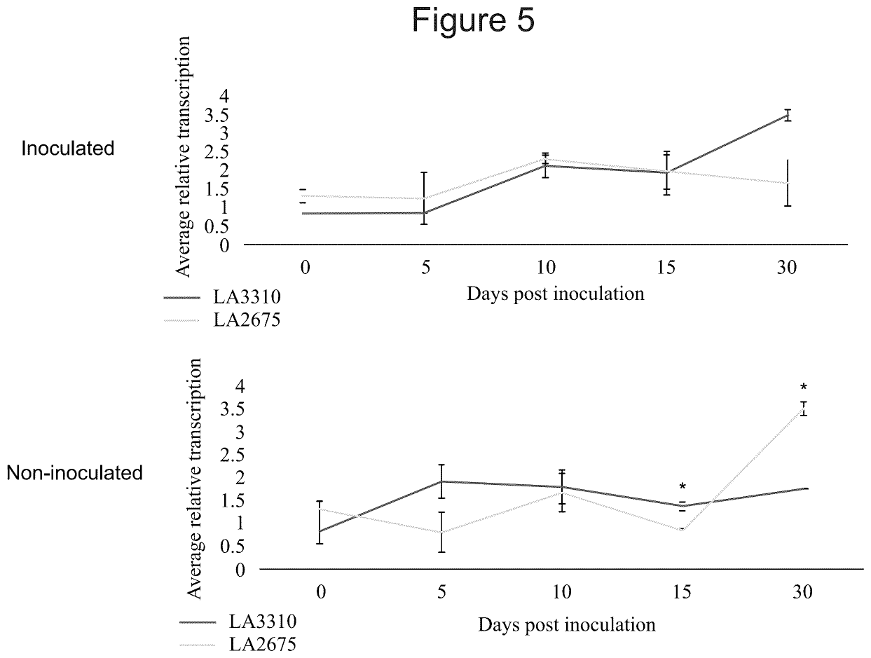

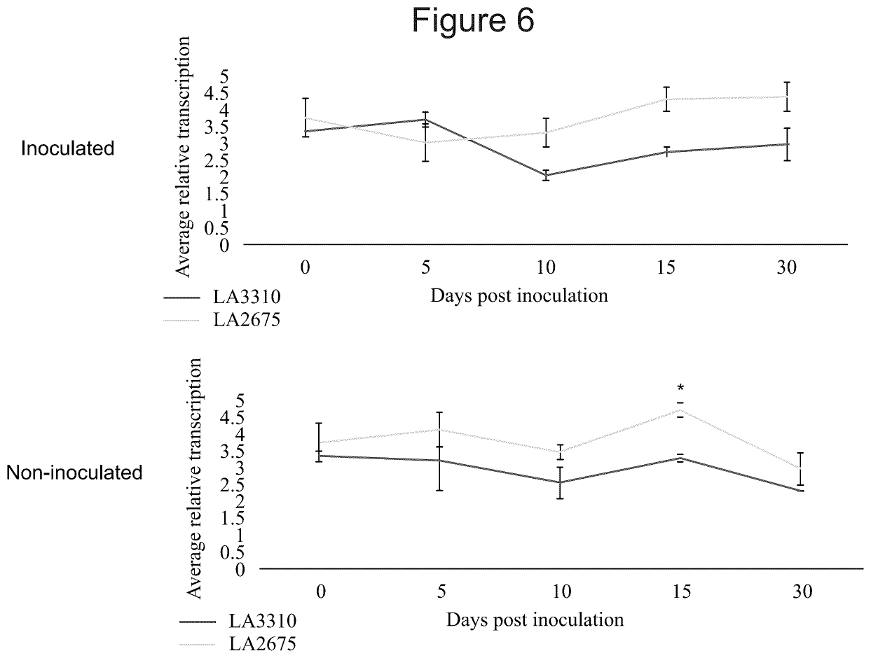

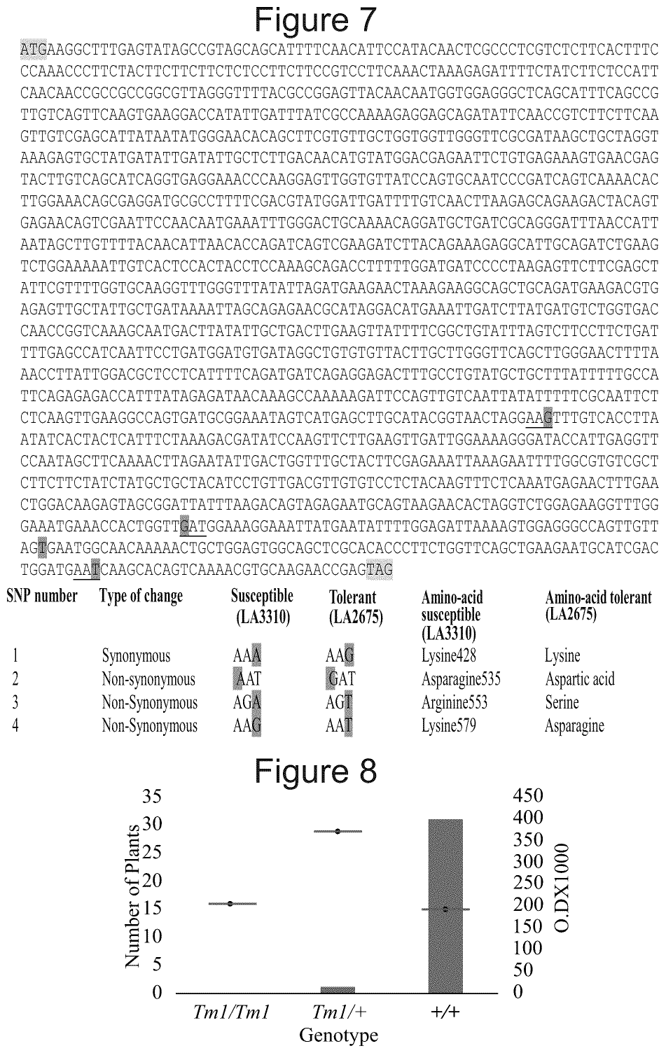

The high throughput transcriptional analysis revealed that Solyc11g018760.1, encoding an unknown protein, was not transcribed in both the tolerant LA2675 and the susceptible genotype. Transcriptional analysis of Solyc11g018740.3.1, encoding an ALBINO3-like protein, showed that no statistically significant difference exists between either inoculated or non-inoculated genotypes at all time points tested (FIG. 4). Transcriptional analysis of Solyc11g018750.2.1, encoding an unknown protein, showed that the differences between the two inoculated lines was statistically insignificant at all time points tested. On the other hand, the relative average transcription of the gene was significantly higher at 15 DPI and significantly lower at 30 DPI in susceptible non-inoculated plants in comparison to the tolerant plants (FIG. 5). Transcriptional analysis of Solyc11g018770.3.1, encoding a polynucleotide adenylyltransferase family protein, with high homology to CCA tRNA nucleotidyltransferase 2 (CCA2-1), showed that the differences between the two inoculated lines were statistically insignificant at all time points tested. On the other hand, the relative average transcription level of the gene was significantly higher in non-inoculated tolerant plants at 15 DPI (FIG. 6).

### Example 9

Validation of CCA tRNA Nucleotidyltransferase 2 (Solyc11g018770.3.1), as the Gene Controlling Tolerance, Using Viral-Induced-Gene-Silencing (VIGS) with TRV

### Experimental Procedures and Results

Liu et al., 2002 has demonstrated that a tobacco rattle virus (TRV)-based vector can be used to induce gene silencing (VIGS) in tomato. Two plasmids were developed, pTRV1 and pTRV2. pTRV1 contains most of the essential viral genes, including the viral RNA-dependent-RNA-polymerase (RdRp) and movement protein (MP). pTRV2 contain the viral capsid protein (CP) and a multi-cloning-site (MDS) for the insertion of additional sequences—the sequences to be silenced. It has been demonstrated that introduction into the MCS of partial sequence of a host gene is enough to induce silencing of that gene. Inoculation of tomato plants with both pTRV1 and pTRV2 results in TRV infection-when the virus spreads systemically, the endogenous gene transcripts, which are homologous to the insert in the viral vector are degraded by post-transcriptional gene silencing (Liu 2002).

Construction of TRV VIGS Vector pTRV2::CCA tRNA Nucleotidyltransferase 2

A vector for silencing the CCA tRNA nucleotidyltransferase 2 (Solyc11g018770.3.1) gene was constructed by insertion of a 408 bp CCA tRNA nucleotidyltransferase 2 gene fragment into the MCS site of TRV RNA2 essentially as described (Liu et al., 2002). PCR was used to generate a 408 bp fragment (from nucleotide no I to no 408) of the gene using forward primer 5′ GACGAATTCATGAAGGCTTTGAGTATAGC 3′ (SEQ ID NO: 58) and reverse primer 5′ GACGGATCCTCG TCCATACATGTTGTCAAG 3′ (SEQ ID NO: 59). These primers contain an added EcoRI site and a BamHI site, respectively. The PCR product was A-T cloned into the plasmid pGEM-T (Stratagene), excised from pGEM-T an EcoRI plus BamHI fragment, and cloned into pTRV2 (Liu et al., 2002) to yield pTRV2::CCA tRNA nucleotidyltransferase.

### Plant Inoculations and Growth Conditions

For VIGS assay, pTRV1 with pTRV2 or with pTRV2::CCA tRNA nucleotidyltransferase 2 were introduced into Agrobacterium tumefaciens strain GV3101. Agrobacterium cultures at O.D.600=0.8 containing pTRV1 or pTRV2 or pTRV2:: CCA tRNA nucleotidyltransferase 2 were mixed in 1:1 ratio. 10 days old tomato plants were agro-infiltrated with the bacterial mixtures using a 1 ml needleless syringe. The tomato plants were grown at 25° C. in a growth chamber under 16 h light/8 h dark cycle.

To investigate the effect of CCA tRNA nucleotidyltransferase 2 on ToBRFV resistance, these tomato plants were inoculated with ToBRFV 14 days after agro-infiltration. The virus was transmitted mechanically—leaves of ToBRFV-infected tomato source plants were ground in 0.01 phosphate buffer (pH 7.0) and applied to carborundum dusted test plants. The carborandum was washed and the test plants were kept in a greenhouse and screened for symptom development and virus level (using ELISA) 30 days after inoculation.

The tomato genotype used in the experiment was LA2825 which contains the ToMV-resistance gene Tm-1. Inoculation of tomato genotypes containing Tm-1 with ToBRFV results in severe disease symptoms (high disease severity index, DSI) and high viral accumulation level. However, the combination of the Tm-1 QTL with the tolerance-inducing gene on chromosome 11 results in resistance—low DSI and low virus accumulation level following inoculation with ToBRFV.

TABLE 8
Response of LA2825 tomato plants to inoculation with ToBRFV following
silencing of the CCA tRNA nucleotidyltransferase gene by VIGS using TRV

| | ToBRFV inoculation | | | | |
|---|---|---|---|---|---|
| | | Plants with symptoms | | | |
| | | No. plants with | | Plants without symptoms | |
| | No. | DSI = 3 | Av. Virus | No. plants | Av. Virus |
| | inoculated | (% infected/ | level | with | level |
| Treatment | plants | inoculated) | (ELISA) | DSI = 0 | (ELISA) |
| pTRV1 + | 7 | 4 (57%) | 550.0 | 3 (43%) | 51.0 |
| pTRV2 | | | | | |
| pTRV1 + | 29 | 2 (6.7%) | 470.0 | 27 (93%) | 7.1 |
| pTRV2::CCA | | | | | |

### REFERENCE

Liu, Y., Schiff, M., Dinesh-Kumar, S. P., 2002. Virus-induced gene silencing. Plant J. 31, 777-786

### Example 10

### Over-Expression of Tm-1

### Introduction

To identify tomato genotypes resistant or tolerant to ToBRFV, 160 genotypes were screened, resulting in the identification of an unexpectedly high number of tolerant genotypes and a single genotype resistant to the virus (Zinger 2021). A selected tolerant genotype (LA2675) and the resistant genotype (CC4173) were further analyzed. Analysis of genetic inheritance revealed that tolerance is controlled by a single recessive gene that was mapped to chromosome 11. Resistance control is mainly recessive and is controlled by two genes. Allelic test between the tolerant and the resistant genotype revealed that these two genotypes share the locus controlling tolerance, mapped to chromosome 11. This locus displayed a strong association with the tolerance trait. This same locus displayed a statistically significant association with symptom levels in segregating populations based on the resistant genotype. However, in these populations, the locus was able to explain only ˜41% of the variation in symptom levels, confirming that additional loci are involved in the genetic control of the resistance trait in this genotype. A locus on chromosome 2, at the region of the Tm-1 gene, was found to interact with the locus discovered on chromosome 11 to control resistance.

### Materials and Methods

### Over-Expression of Tm-1

Tm-1 cDNA (GenBank accession: AB287296.1) also known as Tm-1\hat{ }GCR237 was cloned from S. lycopersicum cultivar LA2825 (Tm1/Tm1) by PCR amplification with the following primers:

Tm1-OE-SalI_Forward
(SEQ ID NO: 119)
5′ GTCGACATGGCAACTGCACAGAG 3′
and
Tm1-OE-NotI_Reverse
(SEQ ID NO: 120)
5′ GCGGCCGCGTCACTCCATAGAAATAG 3′,

containing the Sall site and NotI site, respectively, at their 5′ end (underlined). After sequence validation, Tm-1 cDNA was inserted into a pBIN vector under the control of the 35S promoter. To create a pBIN expression vector, the cassette containing the 35S promoter, omega enhancer, and the NOS terminator was cloned into the HindIII-EcoRI sites of pBINPLUSinto.

Transgenic tomato plants were generated by Agrobacterium tumefaciens-mediated transformation of cotyledons from 14 days old seedlings of LA2675, a ToBRFV tolerant genotype (McCormick, 1991)

### Characterization of Transgenic Plants

To validate incorporation of Tm-1 the over-expression constructs, DNA samples extracted from individual transformed plants served as templates in PCRs using a primer complementary to the 35S promoter (5′-CCTTCGCAAGACCCTTCCTC T-3′ (SEQ ID NO: 121)) and a primer complementary to 3′ of the Tm-1 gene sequence 5′-ACTGAAGGAAACAATACCAAGTCTG-′3 (SEQ ID NO: 122).

Tomato Plants Inoculation with ToBRV and Symptoms Severity Determination

Tomato seedlings were inoculated at first true leaf stage and grown for 30 days in a protected greenhouse. Symptoms severity was determined by a disease severity scale of 0-3; 0-no symptoms, 1-light mosaic, 2-severe mosaic, 3-severe mosaic with leaf narrowing.

### Virus Accumulation

Virus accumulation was assessed by enzyme-linked immunosorbent assay (ELISA) at 30 days post inoculation as described by Koenig, (Koenig, 1981).

### Tm-1 Relative Expression Level

Tm-1 relative expression level in transgenic plants was assessed by Quantitative RT-PCR analysis. Total RNA was extracted from 2-3 small apical leaves with NucleoSpin RNA plant kit. The concentration and integrity of the RNA samples were determined by using an ND1000 spectrophotometer (Thermo Scientific). First-strand cDNA was synthesized from 1 μg of total RNA using qscript cDNA synthesis kit (quanta bio).

Tm-1 primers were designed using the NCBI primer design tool (www (dot)ncbi(dot)nlm(dot)nih(dot)gov/tools/primer-blast/): Tm1qpcr_F=5′-TTCCTCTCCGAGCATGTGAG 3′ (SEQ ID NO: 123) and Tmlqpcr_R=5′-TGGAAGAGATCGGAAGGCAG-3′ (SEQ ID NO: 124), forming a 342 bp amplicon. The qRT-PCR was carried out on a Rotor-Gene 6000 (Qiagen) with the following profile: 40 cycles of 95° C. s, 60° C. 15 s, and 72° C. 20 s; qRT-PCR reactions (12 μl volume) included 3 μl of plant cDNA, 6 μl of ABsolute qPCR mix, SYBR Green, ROX (Thermo Scientific), and 0.125 μM of each primer.

qRT-PCR analyses were performed using the Rotor-Gene Q detection system and data was collected and analyzed with the Rotor-Gene 6000 software version 1.7.28 (Qiagen).

Relative abundance of Tm-1 transcripts were calculated by the AACT (Delta Delta cT) method (Livak, 2001). The abundance of Tm-1 in the tolerant genotype LA2675 was used as reference sample

$$Δ Ct = {Ct}_{Sample} - {Ct}_{sample 18 s}$$

$$ΔΔ Ct = Δ Ct - Δ {Ct}_{reference sample}$$

Relative expression=2\hat{ }(−ΔΔCt)

Where CT represents the fractional cycle number at which the fluorescence crosses a fixed threshold (usually set on 0.05).

### Results

To test the involvement of Tm-1 in ToBRFV resistance, we have crossed the tolerant genotype (LA2675) with the resistant genotype (CC4173) and developed an F3 population segregating for the resistant allele of Tm-1. The F3 plants were inoculated with ToBRFV and analyzed for the correlation between virus accumulation level (ELISA) and the presence of the resistant allele of Tm-1. All of the F3 plants didn't show disease symptoms (DSI=0) as expected from a cross between a resistant donor and a tolerant one. However, it was found that plants homozygote for Tm-1 hardly accumulated any virus, while plants heterozygote for Tm-1 accumulated a low level of virus. This contrasted with plants without Tm-1 which accumulated a very high level of virus (FIG. 8).

TABLE 9
Transgenic tolerant plants over-expressing the resistant
allele of Tm-1 are highly resistant to ToBRFV.

| | ELISA | Presence of | Relative |
|---|---|---|---|
| Genotype | O.D × 1000 | Transgene | expression* |
| LA 2675 | 741.1 | − | 1 |
| Moneymaker | 679.9 | − | 2.6 |
| TM-127 -1 | 0 | + | 908.6 |
| TM-127 -2 | 601.2 | − | 4.8 |
| TM-127 -3 | 0 | + | 743.1 |
| TM-127 -4 | 0 | + | 577.0 |
| TM-127 -5 | 702.95 | − | 1.9 |
| TM-127 -6 | 0 | + | 406.6 |
| TM-127 -7 | 0 | + | 366.5 |
| TM-127 -8 | 0 | + | 35.8 |
| TM-129 -1 | 1026.0 | − | 1.1 |
| TM-129 -2 | 557.0 | − | 19.3 |
| TM-129 -3 | 834.0 | − | 18.4 |
| TM-129 -5 | 662.5 | + | 16.1 |
| TM-130 -1 | 0 | + | 55.4 |
| TM-130 -2 | 580.6 | − | 6.4 |
| TM-130 -3 | 845.6 | − | 9.6 |
| TM-130 -4 | 0 | + | 256.4 |

Relative expression level was analyzed by quantitative RT-PCR, the expression level of the non-transgenic genotype LA2675 was determined as 1.
Next, the resistant allele of Tm-1 was over-expressed in plants of the tolerant genotype (LA2675). The rationale was that if indeed Tm-1 participates in ToBRFV-resistance, its overexpression will “convert” the tolerant genotype (LA2675) from tolerant to resistant to the virus. Plants of 3 T1 transgenic plants were inoculated with ToBRFV and analyzed for symptom development, virus level and Tm-1 expression level. The presence of the Tm-1 transgene was determined by PCR, and its relative expression level was analyzed by quantitative RT-PCR. All the transgenic plants showed no disease symptoms at 30 DPI, again as expected from the tolerant genotype. However, all the segregating T1 plants that expressed a high level of the Tm-1 transgene had accumulated practically zero virus based on ELISA test (Table 9). The transgenic plants that didn't express the transgene, or expressed it at a low level, accumulated a very high level of virus, similar to the accumulation level of the control non-transgenic LA2675 plants, or the accumulation level of the susceptible control Moneymaker plants.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. McCormick S. 1991. Transformation of tomato with Agrobacterium tumefaciens. In: Lindsey K, ed. Plant tissue culture manual, Vol. B6. The Netherlands: Kluwer Academic Publishers, 1-9.
2. Koenig, R. 1981. Indirect ELISA methods for the broad specificity detection of plant viruses. J. Gen. Virol. 55: 53-62.
3. Livak K J, Schmittgen T D. 2001. Analysis of relative gene expression data using real-time quantitative PCR and the 2(−Delta Delta C(T)) Method. Methods. 25(4):402-408. doi: 10.1006/meth.2001.1262
4. Zinger A, Lapidot M, Harel A, Doron-Faigenboim A, Gelbart D, Levin, I. 2021. Identification and Mapping of Tomato Genome Loci Controlling Tolerance and Resistance to Tomato Brown Rugose Fruit Virus. Plants 2021, 10, 179. www(dot)doi(dot)org/10.3390/plants10010179

### REFERENCES

### (Other References are Listed Throughout the Application)

1. Adams, M. J.; Antoniw, J. F.; Kreuze, J. Virgaviridae: a new family of rod-shaped plant viruses. Arch. Virol. 2009, 154, 1967-1972.
2. Alfaro-Fernández, A.; Castillo, P.; Sanahuja, E.; Rodríguez-Salido, M. C.; Font, M. I. First report of Tomato brown rugose fruit virus in tomato in Spain. Plant Dis. 2020, On-line, www(dot)doi(dot)org/10(dot) 1094/PDIS-Jun. 20, 1251-PDN.
3. Alkowni R.; Alabdallah O.; Fadda Z. Molecular identification of tomato brown rugose fruit virus in tomato in Palestine. J. Plant Pathol. 2019, 101, 719-723.
4. Amer, M. A.; Mahmoud, S. Y. First report of Tomato brown rugose fruit virus on tomato in Egypt. New Dis. Reports 2020, 41, 24.
5. Azari; R.; Reuveni; M.; Evenor; D.; Nahon; S.; Shlomo; H.; Chen; L.; Levin; I. Overexpression of UV-DAMAGED DNA BINDING PROTEIN/links plant development and phytonutrient accumulation in high pigment-1 tomato. J Exp Bot. 2010, 61, 3627-3637.
6. Belkhadir, Y.; Subramaniam, R.; Dangl, J. L. Plant disease resistance protein signaling: NBS-LRR proteins and their partners. Curr. Opin. Plant Biol. 2004, 7, 391-399.
7. Benjamini, Y.; Hochberg, Y. Controlling the false discovery rate: A practical and powerful approach to multiple testing. J. R. Stat. Soc. Ser. B 1995, 57, 289-300.
8. Beris, D.; Malandraki, I.; Kektsidou, O.; Theologidis, I.; Vassilakos, N.; Varveri, C. First Report of Tomato Brown Rugose Fruit Virus Infecting Tomato in Greece. Plant Dis. 2020, 104, 2035.
9. Broadbent, L. Epidemiology and control of tomato mosaic virus. Annu. Rev. Phytopathol. 1976, 14, 75-96.
10. Caldwell, J. Persistence of tomato aucuba mosaic virus in dried leaf tissue. Nature 1959, 183, 1142.
11. Camacho-Beltrán, E.; Pérez-Villarreal, A.; Leyva-López, N. E.; Rodríguez-Negrete, E. A.; Ceniceros-Ojeda, E. A.; Méndez-Lozano, J. Occurrence of Tomato brown rugose fruit virus infecting tomato crops in Mexico. Plant Dis. 2019, 103, 1440.
12. Cooper, J. I.; Jones, A. T. Responses of plants to viruses: proposals for the use of terms. Phytopathol. 1983, 73, 127-128.
13. DePristo, M. A.; Banks, E.; Poplin, R.; Garimella, K. V.; Maguire, J. R.; Hartl, C.; Philippakis, A. A.; del Angel, G.; Rivas M. A.; Hanna M.; McKenna A.; Fennell T. J.; Kernytsky A. M.; Sivachenko A. Y.; Cibulskis K.; Gabriel S. B.; Altshuler D.; Daly M. J. A framework for variation discovery and genotyping using next-generation DNA sequencing data. Nat. Genet. 2011, 43, 491-498.
14. Dombrovsky, A.; Sapkota, R.; Lachman, O.; Pearlsman, M.; Antignus, Y. A new aubergine disease caused by a whitefly-borne strain of Tomato mild mottle virus (TomMMoV). Plant Pathol. 2013, 62, 750-759.
15. Fidan, H.; Sarikaya, P.; Calis, O. First report of Tomato brown rugose fruit virus on tomato in Turkey. New Dis. Reports 2019, 39, 18.
16. Fulton, T. M.; Chunwongse, J.; Tanksley S. D. Microprep protocol for extraction of DNA from tomato and other herbaceous plants. Plant Mol. Biol. Rep. 1995, 13, 207-209.
17. Fraser, R. S. S. The genetics of resistance to plant viruses. Annu. Rev. Phytopathol. 1990, 28, 179-200.
18. Gentleman, R. C.; Carey, V. J.; Bates, D. M.; Bolstad, B.; Dettling, M.; Dudoit, S.; Ellis, B.; Gautier, L.; Ge, Y.; Gentry, J.; Hornik, K.; Hothorn, T.; Huber, W.; lacus, S.; Irizarry, R.; Leisch, F.; Li, C.; Macchler, M.; Rossini, A. J.; Sawitzki, G.; Smith, C.; Smyth, G.; Tierney, L.; Yang, J. Y.; Zhang, J. Bioconductor: open software development for computational biology and bioinformatics. Genome Biol., 2004, 5(10):R80.
19. Ishibashi, K.; Masuda, K.; Naito, S.; Meshi, T.; Ishikawa, M. An inhibitor of viral RNA replication is encoded by a plant resistance gene. Proc. Natl. Acad. Sci. U.S.A. 2007, 104, 13833-13838.
20. Kang, B. C.; Yeam, I.; Jahn, M. M. Genetics of plant virus resistance. Annu. Rev. Phytopathol. 2005, 43, 581-621.
21. Kim, D.; Pertea, G.; Trapnell, C.; Pimentel, H.; Kelley, R.; Salzberg, S. L. TopHat2: Accurate alignment of transcriptomes in the presence of insertions, deletions and gene fusions. Genome Biol. 2013, 14, R36.
22. Koenig, R. Indirect ELISA methods for the broad specificity detection of plant viruses. J. Gen. Virol. 1981, 55, 53-62.
23. Kramer, C. Y. Extension of multiple range tests to group means with unequal numbers of replications. Biometrics 1956, 12, 309-310.
24. Lapidot, M.; Karniel, U.; Gelbart, D.; Fogel, D.; Evenor, D.; Kutsher, Y.; Makhbash, Z.; Nahon, S.; Shlomo, H.; Chen, L.; Reuveni, M.; Levin, I. A novel route controlling begomovirus resistance by the messenger RNA surveillance factor pelota. PLOS Genet. 2015 Oct. 8; 11(10):c1005538. doi: 10.1371/journal.pgen.1005538.
25. Lapidot, M.; Levin, I. Genetic resistance to viruses in tomato. In achieving sustainable cultivation of tomatoes; Mattoo, A., Handa, A., Eds., Burleigh Dodds Science Publishing, 2017; pp. 381-400.
26. Lanfermeijer, F. C.; Dijkhuis, J.; Sturre, M. J.; de Haan, P.; Hille, J. Cloning and characterization of the durable tomato mosaic virus resistance gene Tm-2² from Lycopersicon esculentum. Plant Mol. Biol. 2003, 52, 1037-1049.
27. Lewandowski, D. J. Tobamoviruses. In Encyclopedia of Virology (Third Edition); Mahy, B. W. J., Van Regenmortel, M. H. V., Eds., New York: Academic Press Inc., 2008; pp. 68-72.
28. Li, H.; Handsaker, B.; Wysoker, A.; Fennell, T.; Ruan J.; Homer, N.; Marth, G.; Abecasis, G.; Durbin, R.; 1000 Genome Project Data Processing Subgroup. The Sequence Alignment/Map format and SAMtools. Bioinformatics 2009, 25, 2078-2079.
29. Li, H.; Durbin, R. Fast and accurate long-read alignment with Burrows-Wheeler transform. Bioinformatics 2010, 26, 589-595
30. Ling, K.-S.; Tian, T.; Gurung, S.; salati, R.; Gilliard, A. First report of tomato brown fruit rugose virus infecting greenhouse tomato in the United States. Plant Dis. 2019, 103, 1439.
31. Luria, N.; Smith, E.; Reingold, V.; Bekelman, I.; Lapidot, M.; Levin, I.; Elad, N.; Tam, Y.; Scla, N.; Abu-Ras, A.; Ezra, N.; Haberman, A.; Yitzhak, L.; Lachman, O.; Dombrovsky, A. A new Israeli tobamovirus isolate infects tomato plants harboring Tm-2² resistance genes. PLOS One 2017, 12(1):c0170429. doi: 10.1371/journal.pone.0170429.
32. Maayan, Y.; Pandaranayaka, E. P. J.; Srivastav,a D. A.; Lapidot, M.; Levin, I.; Dombrovsky, A.; Harel, A. Using genomic analysis to identify tomato Tm-2 resistance-breaking mutations and their underlying evolutionary path in a new and emerging tobamovirus. Arch. Virol. 2018, 163, 1863-1875.
33. Menzel, W.; Knierim, D.; Winter, S.; Hamacher, J.; Heupel, M. First report of tomato brown fruit rugose virus infecting tomato in Germany. New Dis. Reports 2019, 39, 1.
34. Morse, J. W.; Deutscher M. P. A physiological role for tRNA nucleotidyltransferase during bacteriophage infection. Biochem Biophys Res Commun. 1976, 73, 953-959.
35. Nicaise, V. Crop immunity against viruses: outcomes and future challenges. Front. Plant Sci. 2014, 5, 660.
36. Nunes, A.; Ribeiro, D. R.; Marques, M.; Santos, M. A. S.; Ribeiro, D.; Soares, A. R. Emerging Roles of tRNAs in RNA Virus Infections. Trends Biochem Sci. 2020, 45:794-805.
37. Panno, S.; Caruso, A. G.; Davino, S. First report of tomato brown fruit rugose virus on tomato crops in Italy. Plant Dis. 2019, 103, 1443.
38. Paudel, D. B.; Sanfaçon, H. Exploring the diversity of mechanisms associated with plant tolerance to virus infection. Front Plant Sci. 2018, 9, 1575.
39. Pelham, J. Resistance in tomato to Tobacco mosaic virus. Euphytica 1966, 15, 258-267.
40. Robaglia, C.; Caranta, C. Translation initiation factors: a weak link in plant RNA virus infection. Trends Plant Sci. 2006, 11, 40-45.
41. Robinson, M. D.; Mccarthy, D. J.; Smyth, G. K. edgeR: a bioconductor package for differential expression analysis of digital gene expression data. Bioinformatics 2010, 26, 139-40.
42. Salem, N.; Mansour, A.; Ciuffo, M.; Falk, B. W., Turina, M. A new tobamovirus infecting tomato crops in Jordan. Arch Virol. 2016, 161, 503-506.
43. Sastry, S. K; Zitter, T. A. Management of virus and viroid diseases of crops in the tropics. In Plant Virus and Viroid Diseases in the Tropics, Vol. 2 Epidemiology and Management; Springer: Dordrecht, The Netherlands, 2014; pp. 149-480.
44. Soyk, S.; Lemmon, Z. H.; Oved, M.; Fisher, J.; Liberatore, K. L.; Park, S. J.; Goren, A.; Jiang, K.; Ramos, A.; van der Knaap, E.; Van Eck, J.; Zamir, D.; Eshed, Y.; Lippman, Z. B. Bypassing negative epistasis on yield in tomato imposed by a domestication gene. Cell 2017, 169, 1142-1155.
45. Sundberg, E.; Slagter, J. G.; Fridborg, I.; Cleary, S. P.; Robinson, C.; Coupland, G. ALBINO3, an Arabidopsis nuclear gene essential for chloroplast differentiation, encodes a chloroplast protein that shows homology to proteins present in bacterial membranes and yeast mitochondria. Plant Cell 1997, 9, 717-730.
46. Trapnell, C.; Williams, B. A.; Pertea, G.; Mortazavi, A.; Kwan, G.; van Baren, M. J.; Salzberg, S. L.; Wold, B. J.; Pachter, L. Transcript assembly and quantification by RNA-Seq reveals unannotated transcripts and isoform switching during cell differentiation. Nat. Biotechnol. 2010, 28, 511-515.
47. Vörtler, S.; Mörl, M. tRNA-nucleotidyltransferases: highly unusual RNA polymerases with vital functions. FEBS Lett. 2010, 584, 297-302.
48. Walkey, D. G. A. 1985. Applied plant virology. Wiley-Interscience, New York, 1985; p. 236-242.
49. Watanabe, Y.; Kishibayashi, N.; Motoyoshi, F.; Okada, Y. Characterization of Tm-1 gene action on replication of common isolates and a resistance-breaking isolate of TMV. Virology 1987, 161, 527-532.
50. Weber, E.; Engler, C.; Gruetzner, R.; Werner, S.; Marillonnet, S. A modular cloning system for standardized assembly of multigene constructs. PLOS One 2011, 6(2):e16765. doi: 10.1371/journal.pone.0016765. PMID: 21364738; PMCID: PMC3041749.
51. Wesley, S. V.; Helliwell, C. A.; Smith, N. A.; Wang, M. B.; Rouse, D. T.; Liu, Q.; Gooding, P. S.; Singh, S. P.; Abbott, D.; Stoutjesdijk, P. A.; Robinson, S. P.; Gleave, A. P.; Green, A. G.; Waterhouse, P. M. Construct design for efficient, effective and high-throughput gene silencing in plants. Plant J. 2001 27, 581-590.
52. Yan, Z.; Ma, H.; Han, S.; Li, X. First Report of Tomato brown rugose fruit virus Infecting Tomato in China. Plant Dis. 2019, 103, 11.
53. Young. N. D.; Tanksley. S. D. RFLP analysis of the size of chromosomal segments retained around the Tm-2 locus of tomato during backcross breeding. Theor. Appl. Genet. 1989, 77, 353-359.
54. Zhao. J; Zhang. X: Hong. Y; Liu. Y. Chloroplast in Plant-Virus Interaction. Front Microbiol. 2016, 7, 1565. doi: 10.3389/fmich.2016.01565.

What is claimed is:

1. A method of producing a tomato plant exhibiting tolerance to ToBRFV, comprising:
identifying a tomato plant exhibiting tolerance to ToBRFV by marker assisted selection, wherein a marker of said marker assisted selection is selected from the group consisting of Eco105I_9.42, AciI_9.47 and NcoI_9.53.

2. A method of producing a tomato plant exhibiting tolerance to ToBRFV, comprising:
identifying a tomato plant exhibiting tolerance to ToBRFV by marker assisted selection, wherein a marker of said marker assisted selection is in a gene set forth in Solyc11g018770.3.1.

3. A method of producing a tomato plant exhibiting tolerance to ToBRFV, the method comprising downregulating expression Solyc11g018770.3.1 in a cell of the tomato plant optionally wherein the tomato is an open-field tomato, thereby conferring tolerance to ToBRFV.

4. A method of producing a tomato plant exhibiting resistance to ToBRFV, the method comprising, producing a tomato plant exhibiting tolerance to ToBRFV according to the method of claim 1, wherein said tomato plant comprises in its genome a Tm-1 resistance gene and/or Tm-2 resistance gene, and/or a QTL2 on chromosome 9.

5. A tomato plant comprising in its genome a nucleic acid sequence variation in a homozygous form in at least one gene set forth in Solyc11g018770.3.1 resulting in tolerance to Tomato Brown Rugose Fruit virus (TOBRFV).

6. The plant of claim 5, further comprising in its genome a Tm-1 resistance gene on chromosome 2 and/or a QTL2 on chromosome 9.

7. The plant of claim 4, wherein said Tm-1 resistance gene and/or Tm-2 resistance gene is in a heterozygous form.

8. A method of breeding tomato, the method comprising:
(a) providing the plant of claim 5; and
(b) subjecting it to a breeding program.

9. A method of producing tomato fruits, the method comprising:
(a) growing the plant of claim 5 to the stage of development of fruits; and
(b) harvesting the fruits.

10. A method of processing tomato comprising:
(a) providing tomato fruit of the plant of claim 5; and
(b) processing said fruit.

11. A method for improving the yield of tomato plants in an environment infested by ToBRFV or likely to be infection by ToBRFV or reducing the loss on tomato production in condition of ToBRFV infestation or protecting a field, tunnel, greenhouse or glasshouse of tomato plants from TOBRFV infestation, the method comprising growing tolerant or resistant tomato plants of claim 5.

12. An edible processed product of the plant of claim 5.

13. The processed product of claim 12 selected from the group consisting of a tomato paste, a ketchup, a tomato sauce a tomato soup, a tomato juice, a tomato powder, a tomato dice, a crushed tomato, a chopped tomato and a tomato concentrate.

14. A modified gene comprising a null mutation which confers resistance to ToBRFV, wherein said modified gene comprises a nucleic acid sequence having at least 90% identity with respect to the cultivated allele of Solyc11g018770.3.1 (SEQ ID NO:13).

15. A cultivated tomato plant containing a modified gene according to claim 14.

16. A probe or primer pair for identifying a marker for a tomato plant exhibiting tolerance to ToBRFV by marker assisted selection, wherein a marker of said marker assisted selection is in a gene set forth in Solyc11g018770.3.1.

17. A method for protecting a field, tunnel or glasshouse of tomato plants from ToBRFV infestation, comprising of growing resistant or tolerant tomato plants to ToBRFV as defined in claim 5.

18. A method for increasing the number of harvestable or viable tomato plants in an environment infested by ToBRFV comprising growing tomato plants resistant or tolerant to ToRBFV as defined in claim 5.
